SEQUENCE LISTING (20 sequences)

SEQ ID NO: 1 - DNA, 4545 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagctgaaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 2 - DNA, 5509 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatggctacccaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactggttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 3 - DNA, 4859 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatggagcgctgccccagcctaggggtcaccctctacgccctggtggtggtcctggggctgcgggcgacaccggccggcggccagcactatctccacatccgcccggcacccagcgacaacctgcccctggtggacctcatcgaacacccagaccctatctttgaccccaaggaaaaggatctgaacgagacgctgctgcgctcgctgctcgggggccactacgacccaggcttcatggccacctcgccccccgaggaccggcccggcgggggcgggggtgcagctgggggcgcggaggacctggcggagctggaccagctgctgcggcagcggccgtcgggggccatgccgagcgagatcaaagggctagagttctccgagggcttggcccagggcaagaagcagcgcctaagcaagaagctgcggaggaagttacagatgtggctgtggtcgcagacattctgccccgtgctgtacgcgtggaacgacctgggcagccgcttttggccgcgctacgtgaaggtgggcagctgcttcagtaagcgctcgtgctccgtgcccgagggcatggtgtgcaagccgtccaagtccgtgcacctcacggtgctgcggtggcgctgtcagcggcgcgggggccagcgctgcggctggattcccatccagtaccccatcatttccgagtgcaagtgctcgtgctagaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccaccactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 4 - DNA, 4213 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatgaaccggaaagcgcggcgctgcctgggccacctctttctcagcctgggcatggtctacctccggatcggtggcttctcctcagtggtagctctgggcgcaagcatcatctgtaacaagatcccaggcctggctcccagacagcgggcgatctgccagagccggcccgacgccatcatcgtcataggagaaggctcacaaatgggcctggacgagtgtcagtttcagttccgcaatggccgctggaactgctctgcactgggagagcgcaccgtcttcgggaaggagctcaaagtggggagccgggaggctgcgttcacctacgccatcattgccgccggcgtggcccacgccatcacagctgcctgtacccagggcaacctgagcgactgtggctgcgacaaagagaagcaaggccagtaccaccgggacgagggctggaagtggggtggctgctctgccgacatccgctacggcatcggcttcgccaaggtctttgtggatgcccgggagatcaagcagaatgcccggactctcatgaacttgcacaacaacgaggcaggccgaaagatcctggaggagaacatgaagctggaatgtaagtgccacggcgtgtcaggctcgtgcaccaccaagacgtgctggaccacactgccacagtttcgggagctgggctacgtgctcaaggacaagtacaacgaggccgttcacgtggagcctgtgcgtgccagccgcaacaagcggcccaccttcctgaagatcaagaagccactgtcgtaccgcaagcccatggacacggacctggtgtacatcgagaagtcgcccaactactgcgaggaggacccggtgaccggcagtgtgggcacccagggccgcgcctgcaacaagacggctccccaggccagcggctgtgacctcatgtgctgtgggcgtggctacaacacccaccagtacgcccgcgtgtggcagtgcaactgtaagttccactggtgctgctatgtcaagtgcaacacgtgcagcgagcgcacggagatgtacacgtgcaagtgaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 5 - DNA, 32 bp, Artificial Sequence
  Oligonucleotide SHH_F
aggatccaccatgctgctgctggcgagatgtc

SEQ ID NO: 6 - DNA, 30 bp, Artificial Sequence
  Oligonucleotide SHH_R
tataagctttcagctggacttgaccgccat

SEQ ID NO: 7 - DNA, 20 bp, Artificial Sequence
  Oligonucleotide SHH_SF
ttatccccaatgtggccgag

SEQ ID NO: 8 - DNA, 20 bp, Artificial Sequence
  Oligonucleotide SHH_FR
ctgagtcatcagcctgtccg

SEQ ID NO: 9 - DNA, 34 bp, Artificial Sequence
  Oligonucleotide CTNNB_F
atcgtcgaccaccatggctacccaagctgatttg

SEQ ID NO: 10 - DNA, 31 bp, Artificial Sequence
  Oligonucleotide CTNNB_R
ttcggtaccttacaggtcagtatcaaaccag

SEQ ID NO: 11 - DNA, 20 bp, Artificial Sequence
  Oligonucleotide CTNNB_SF
atgactcgagctcagagggt

SEQ ID NO: 12 - DNA, 20 bp, Artificial Sequence
  Oligonucleotide CTNNB_SR
attgcacgtgtggcaagttc

SEQ ID NO: 13 - DNA, 26 bp, Artificial Sequence
  Oligonucleotide NOG_F
ggatccaccatggagcgctgccccag

SEQ ID NO: 14 - DNA, 28 bp, Artificial Sequence
  Oligonucleotide NOG_R
atagaattctagcacgagcacttgcact

SEQ ID NO: 15 - DNA, 20 bp, Artificial Sequence
  Oligonucleotide NOG_SF
gatctgaacgagacgctgct

SEQ ID NO: 16 - DNA, 20 bp, Artificial Sequence
  Oligonucleotide NOG_SR
tagccctttgatctcgctcg

SEQ ID NO: 17 - DNA, 35 bp, Artificial Sequence
  Oligonucleotide WNT_F
atcgtcgaccaccatgaaccggaaagcgcggcgct

SEQ ID NO: 18 - DNA, 33 bp, Artificial Sequence
  Oligonucleotide WNT_R
ttcggtacctcacttgcacgtgtacatctccgt

SEQ ID NO: 19 - DNA, 20 bp, Artificial Sequence
  Oligonucleotide WNT_SF
gcgacaaagagaagcaaggc

SEQ ID NO: 20 - DNA, 20 bp, Artificial Sequence
  Oligonucleotide WNT_SR
ctcctccaggatctttcggc

(19) United States
(12) Patent Application Publication
(10) Pub. No.: US 2024/0060083 A1
(43) Pub. Date: Feb. 22, 2024

(54) Gene therapy DNA vector based on gene therapy DNA vector VTvaf17 carrying the therapeutic gene selected from the group of SHH, CTNNB1, NOG, and WNT7A genes for increasing the expression level of these therapeutic genes, method of its production and use, Escherichia coli strain SCS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A carrying the gene therapy DNA vector, method

(71) Applicants: CELL AND GENE THERAPY LTD, Rostov-on-Don (GB); OBSCHESTVO S OGRANICHENNOI OTVETSTVENNOSTJU "PRORYVNYE INNOVATSIONNYE TEKHNOLOGII", Moscow (RU)

(72) Inventor: Natalia SAVELIEVA, Wienna (AT)

(21) Appl. No.: 18/268,887

(22) PCT Filed: Dec. 18, 2019
(86) PCT No.: PCT/RU2019/000968

§ 371 Date: Jun. 21, 2023

(30) Foreign Application Priority Data
Dec. 21, 2018 (RU) 2018145686

(51) Int. Cl. C12N 15/85; C12N 15/70; C12N 1/20

(52) U.S. Cl. CPC C12N 15/85; C12N 15/70; C12N 1/205; C12R 2001/19

(57) ABSTRACT

The invention refers to genetic engineering and can be used in biotechnology, medicine, and agriculture for the manufacture of gene therapy products. A gene therapy DNA vector based on the VTvaf17 gene therapy DNA vector is proposed that carries a target gene selected from the group of SHH, CTNNB1, NOG, WNT7A genes for the treatment of diseases characterized by impaired tissue regeneration, wound healing, growth, pigmentation and hair coloring, formation and maturation of hair follicles, processes of differentiation and growth of cells, leading to a decrease in the activity of hair follicles, including with allopecia, autoimmune diseases, hereditary and acquired pathological conditions thawing, and for accelerated healing of wounds, restoration of the hairline and the prevention and inhibition of alopecia. Moreover, the gene therapy DNA vector VTvaf17-SHH, or VTvaf 17 -CTNNB 1, or VTvaf17-NOG, or VTvaf17-WNT7A has the nucleotide sequence of SEQ ID No. 1 or SEQ ID No. 2 or SEQ ID No. 3 or SEQ ID No. 4, respectively. Also provided are a method of producing said vector, the use of a vector, a strain of Escherichia coli carrying said vector, as well as a method of industrial production of said vector.

### FIELD OF THE INVENTION

The invention refers to genetic engineering and can be used in biotechnology, medicine, and agriculture for the manufacture of gene therapy products.

### BACKGROUND OF THE INVENTION

Gene therapy is an innovative approach in medicine aimed at treating inherited and acquired diseases by means of delivery of new genetic material into a patient's cells to compensate for or suppress the function of a mutant gene and/or treat a genetic disorder. The final product of gene expression may be an RNA molecule or a protein molecule. However, most physiological processes in the body are associated with the functional activity of protein molecules, while RNA molecules are either an intermediate product in the synthesis of proteins or perform regulatory functions. Thus, the objective of gene therapy in most cases is to inject the organism with genes that provide transcription and further translation of protein molecules encoded by these genes. Within the description of the invention, gene expression refers to the production of a protein molecule with amino acid sequence encoded by this gene.

SHH, CTNNB1, WNT7A, and NOG genes (alternative name NOGG) included in the group of genes play a key role in several processes in human and animal organisms. The correlations between low/insufficient concentrations of these proteins and different adverse states in some cases confirmed by disturbances in normal gene expression encoding these proteins was demonstrated. Thus, the gene therapy upregulation of expression of a gene selected from the group of SHH, CTNNB1, WNT7A, and NOG genes (alternative name NOGG) has potential to correct various conditions in humans and animals.

The SHH gene (another gene name is HPE3) encodes the SHH (Sonic Hedgehog) protein that is necessary for embryogenesis and regulates cell growth, differentiation and normal generation of organs, including spatial generation. This protein is important for the development of brain and spinal cord (central nervous system), eyes, limbs, and other body parts.

Sonic Hedgehog is necessary for the development of anterior cortex and is involved in the spatial separation of the right and left sides of forebrain (formation of the ventral surface midline). Furthermore, Sonic Hedgehog and other signalling proteins also regulate the formation of the right and left halves (hemispheres) of the brain and a pair of eyes. SHH gene mutations and insufficient SHH protein activity in the embryogenesis lead to holoprosencephaly, i.e. severe impairment in brain development, in which complete or partial lack of separation of the hemispheres in combination with other malformations of head and face is recorded. Generalised convulsions and other disorders are observed. Although most of these patients die in utero or in infancy, some patients, usually heterozygous for mutations in SHH gene, display less marked symptoms and survive to adulthood subject to appropriate treatment (Weiss K et al.//Genet Med. 2018 January;20(1):14-23).

Other hereditary diseases caused by mutations in the SHH gene include microphthalmia and coloboma that are accompanied by isolated or combined cleavage of iris, retina, choroid, optic nerve, or eyelid. In these pathologies, correction is possible with preservation of vision, productivity, and quality of life of patients (Reis L M, Semina E V//Birth Defects Res C Embryo Today. 2015 June;105(2):96-113).

The described syndromes are associated with disorders that develop during postnatal development. Gene therapy approaches to the prevention and maintenance therapy of such hereditary diseases are currently under development, but perhaps in combination with genome editing technologies will be implemented in the future (Ma H et al.//Nature. 2017 August 24;548(7668):413-419).

Reduced SHH expression can be caused by somatic mutations or dysregulation that are not directly associated with protein coding gene sequence. This may result in various diseases, pathological and deficient body conditions the correction of which is possible, including via gene therapy approach. For example, the SHH gene is involved in myogenesis, including the repair and recovery of muscle tissue damage, and a gene therapy approach using a DNA vector expressing the SHH gene resulted in improved myocardial infarction healing in mice (Roncalli J et al.//Journal of the American College of Cardiology. 2011;57(24):2444-2452). Furthermore, cell-based therapy using CD34+ cells that overexpress SHH also led to improved healing of myocardial injuries in animals (Ahmed et al.//PLoS ONE, 2010, 5(1), e8576).

Intracutaneous injection of gene therapy DNA vector pCS2-SHH expressing SHH into mice accelerated wound healing and promoted angiogenesis in the injury site (Park H J et al.//Biomaterials. 2012 December;33(35):9148-56).

The hair follicle formation and activity thereof is also regulated by SHH gene. SHH knockout mice feature complete absence of hair, and SHH transgene expression restores animal hair (Cui C Y et al.//Cell Cycle. 2011 Oct. 1;10(19):3379-86). Topical applications of SHH agonist activated hair follicles in mice, which is the basis for the development of drugs based on SHH for alopecia treatment in humans (Paladini R D et al.//J Invest Dermatol.2005 October; 125(4): 638-46).

CTNNB1 gene encodes a protein, namely catenin beta 1 (or beta catenin), that is involved in the adhesion formation of epithelial cells and integrity of epithelial tissues. Several clinical case histories of de novo mutations of this gene resulting in the loss of expression thereof have been described. The phenotype of these patients features postnatal microcephaly and several neurodegenerative disorders (Kharbanda M et al.//Eur J Med Genet. 2017 February;60(2):130-135).

Gene expression is also significantly reduced in haemorrhagic post-stroke plaques, which may indicate the role of CTNNB1 in the pathogenesis of blood-brain barrier disorders by reducing the adhesive functions of endothelial cells (Tran A et al.//Circulation. 2016 Jan. 12;133(2):177-86).

Transcriptomic analysis in patients with osteoporosis revealed a correlation of reduced expression of CTNNB1 gene with impaired osteoblast differentiation and bone formation (Zhang Y et al.//Eur Rev Med Pharmacol Sci. 2016;20(3):433-40). Beta-catenin is considered as one of the main therapeutic molecules in pseudoarthrosis (Ghadakzadeh S et al.//FASEB J. 2016 September;30(9):3227-37).

Some CTNNB1 mutations are associated with cancerous tumours, in particular, the CTNNB1 gene deletion is typical for mesotheliomas, and transfection with DNA vector expressing this gene restores the normal cell phenotype in vitro (Usami N et al.//Oncogene. 2003 Sep. 11;22(39):7923-30).

It was shown in another experimental study that CTNNB1 expression protects cells from metabolic stress in renal ischemia (Wang Z et al.//J Am Soc Nephrol. 2009 September;20(9): 1919-28).

A gene therapy approach using a recombinant adenoviral vector expressing the CTNNB1 gene has shown its effectiveness in tissue healing after myocardial infarction due to the protection and activation of both myocyte cells and fibroblasts in the injury site (Hahn J Y et al.//J Biol Chem. 2006 Oct. 13;281(41):30979-89).

In the study of differentiation and growth of hair cells of the organ of Corti that act as the main component of the hearing and vestibular apparatus of all mammals, it was shown that the CTNNB1 expression is necessary for the regulation of Wnt1 and Atoh1 transcription factors. Catenin beta 1 plays a key role in the formation of progenitor cells of hair cells, and without CTNNB1 expression, the formation thereof does not occur in mice (Shi F et al.//J Neurosci. 2014 May 7;34(19):6470-9). In vivo approach including introduction of one-time CTNNB1 mutation that increases the stability of catenin beta 1 protein and concentration thereof in the tissue, resulted in an increase in the number of hair cells in mice and can be considered as the basis for developing therapeutic agents for correcting dysfunctions of hearing and vestibular apparatus (Yeh W H et al.//Nat Commun. 2018 Jun. 5;9(1):2184).

Interestingly, pigmentation and hair colouring also depend on the CTNNB1 expression and, in experiments on mice, it was shown that animals with different hair colour may be bred by affecting CTNNB1 expression level (Enshell-Seijffers D et al.//Proc Natl Acad Sci U S A. 2010 Dec. 14;107(50):21564-9).

Formation and maturation of hair follicles also involves catenin beta 1. Although data on the pathogenetic role of CTNNB1 expression in hair loss, including alopecia, are ambiguous (Fiuraskova M et al.//Arch Dermatol Res. 2005 September;297(3):143-6), clinical trials of some molecules that stimulate catenin beta 1 have proved to be effective in increasing both the number and the quantity of hairs (Tosti A et al.//J Cosmet Dermatol. 2016 December;15(4):469-474).

The NOG gene (alternative name NOGG) encodes the secreted NOG protein that acts as a pleiotropic factor. NOG protein is involved in the formation of neural tube, teeth, hair follicles, eyes, bones, and joints. NOG knockout animals die during early embryogenesis, however, mutations associated with various human and animal diseases are known. For example, proximal symphalangism (featuring fusion of phalanges) is one of the phenotypic manifestations of mutations in the NOG gene. Another manifestation of NOG gene mutations is hearing loss at an early age due to ankylosis of ear bones. Interestingly, topical applications of the NOG protein can change the dental phenotype, causing the growth of molars instead of incisors (Tucker A S, Matthews K L, Sharpe P T//Science 1998;282:1136-8).

Insufficient NOG expression is associated with an imbalance of cells that form bone tissue in patients with ankylosing spondylitis (Xie Z et al.//Arthritis Rheumatol. 2016 February;68(2):430-40). Furthermore, the possibility of prevention of bone resynostosis after surgery was shown using genetically modified cells expressing the NOG gene (Cooper G M et al.//Plast Reconstr Surg. 2009 February;123(2 Suppl):945-1035). Similar results in preventing excessive ossification were obtained using a recombinant adenoviral vector expressing the NOG gene (Glaser et al.//48th Annual Meeting of the Orthopaedic Research Society; Poster No: 0493).

In experimental study on mice, it was shown that the injection of recombinant NOG protein accelerates angenesis and restoration of normal brain function after ischemic stroke (Shin J A et al.//Brain Behav Immun. 2014 August;40:143-54).

In the skin tissues the NOG expression is necessary for activating the growth of hair follicles, and molecular mechanism of this process is associated, among other factors, with an increase in the expression of SHH gene described above (Botchkarev V A et al.//FASEB J. 2001 October;15(12):2205-14). In a small clinical experiment in women with alopecia, it was shown that topical applications of a mixture of growth factors, including the NOG protein followed by microneedle therapy have resulted in clinically significant results, as well as increasing patient satisfaction (Lee Y B et al.//J Dermatol. 2013 January;40(1):81-3).

In an experimental model of Huntington's disease in rats, the use of an adeno-associated viral vector expressing the NOG gene stimulated neuronal growth and slowed the progression of this disease (Benraiss A et al.//Gene Ther. 2012 May;19(5):483-93).

The WNT7A gene encodes a secreted signalling WNT7A protein. This gene is a member of the WNT gene family that is involved in carcinogenesis and in some developmental processes of the organism, including regulation of cell differentiation and patterning during embryogenesis. The WNT7A gene is also involved in the development of the anterior-posterior axis during the formation of female reproductive tract and plays a key role in the functional activity of uterine smooth muscles. Mutations in this gene are associated with Fuhrmann and Al-Awadi/Raas-Rothschild/Schinzel phocomelia syndromes and are also involved in the pathogenesis of certain diseases and adverse conditions.

During the analysis of intestinal biopsy samples affected by ulcerative colitis, it was found that the WNT7A expression is significantly reduced compared to tissue samples from normal sites (You J et al.//Dig Dis Sci. 2008 April;53(4):1013-9).

In Duchenne muscular dystrophy, the WNT7A protein injection increased the functional activity and improved the morphological characteristics of the muscle fibres (von Maltzahn J et al.//Proc Natl Acad Sci U S A. 2012 Dec. 11;109(50):20614-9). Another study has shown that the injection of muscles with cells stimulated by WNT7A an increase in muscle mass and functional activity is observed (Bentzinger C F et al.//J Cell Biol. 2014 April 14;205(1):97-111).

The hair follicle formation in the cicatrisation of wounds is greatly enhanced by overexpression of WNT7A (Ito et al.//Nature, vol. 447, no. 7142, pp. 316-320,2007). Another study has shown that the injection of cells expressing WNT7A into the wound area accelerated wound healing and regeneration of hair growth due to changes in the cell microenvironment and intercellular communications (Dong et al.//Stem Cells Int. 2017;2017:3738071).

Thus, the background of the Invention suggests that mutations in SHH, CTNNB1, NOG, and WNT7A genes or insufficient expression of proteins encoded by these genes are associated with the development of a spectrum of diseases, including, but not limited to, alopecia, autoimmune diseases, hereditary and acquired pathological conditions such as connective tissue damage, and other processes. This is why SHH, CTNNB1, NOG, and WNT7A genes are grouped within this patent. Genetic constructs that provide expression of proteins encoded by SHH, CTNNB1, NOG, and WNT7A genes can be used to develop drugs for the prevention and treatment of different diseases and pathological conditions.

Moreover, these data suggest that insufficient expression of proteins encoded by SHH, CTNNB1, NOG, and WNT7A genes included in the group of genes is associated not only with pathological conditions, but also with a predisposition to their development. Also, these data indicate that insufficient expression of these proteins may not appear explicitly in the form of a pathology that can be unambiguously described within the framework of existing clinical practice standards (for example, using the ICD code), but at the same time cause conditions that are unfavourable for humans and animals and associated with deterioration in the quality of life.

Analysis of approaches to increase the expression of therapeutic genes implies the practicability of use of different gene therapy vectors.

Gene therapy vectors are divided into viral, cell, and DNA vectors (Guideline on the quality, non-clinical, and clinical aspects of gene therapy medicinal Products EMA/CAT/80183/2014). Recently, gene therapy has paid increasingly more attention to the development of non-viral gene delivery systems with plasmid vectors topping the list. Plasmid vectors are free of limitations inherent in cell and viral vectors. In the target cell, they exist as an episome without being integrated into the genome, while producing them is quite cheap, and there is no immune response or side effects caused by the administration of plasmid vectors, which makes them a convenient tool for gene therapy and prevention of the genetic diseases (DNA vaccination) (Li L, Petrovsky N.//Expert Rev Vaccines. 2016;15(3):313-29).

However, limitations of plasmid vectors use in gene therapy are: 1) presence of antibiotic resistance genes for the production of constructs in bacterial strains; 2) the presence of various regulatory elements represented by sequences of viral genomes; 3) length of therapeutic plasmid vector that determines the efficiency of vector delivery to the target cell.

It is known that the European Medicines Agency deems it necessary to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies). This recommendation is primarily related to the potential danger of the DNA vector penetration or horizontal antibiotic resistance gene transfer into the cells of bacteria found in the body as part of normal or opportunistic microflora. Furthermore, the presence of antibiotic resistance genes significantly increases the length of DNA vector, which reduces the efficiency of its penetration into eukaryotic cells.

It is important to note that antibiotic resistance genes also make a fundamental contribution to the method of production of DNA vectors. If antibiotic resistance genes are present, strains for the production of DNA vectors are usually cultured in medium containing a selective antibiotic, which poses risk of antibiotic traces in insufficiently purified DNA vector preparations. Thus, production of DNA vectors for gene therapy without antibiotic resistance genes is associated with the production of strains with such distinctive feature as the ability for stable amplification of therapeutic DNA vectors in the antibiotic-free medium.

In addition, the European Medicines Agency recommends avoiding the presence of regulatory elements in therapeutic plasmid vectors to increase the expression of therapeutic genes (promoters, enhancers, post-translational regulatory elements) that constitute nucleotide sequences of genomes of various viruses (Draft Guideline on the quality, non-clinical and clinical aspects of gene therapy medicinal products, http://www.ema.europa.eu/docs/en_GB/document_library/Scientific_guideline/2015/05/WC500187020.pdf). Although these sequences can increase the expression level of the therapeutic transgene, however, they pose risk of recombination with the genetic material of wild-type viruses and integration into the eukaryotic genome. Moreover, the relevance of overexpression of the particular gene for therapy remains an unresolved issue.

The size of the therapy vector is also essential. It is known that modern plasmid vectors often have unnecessary, non-functional sites that increase their length substantially (Mairhofer J, Grabherr R.//Mol Biotechnol. 2008.39(2):97-104). For example, ampicillin resistance gene in pBR322 series vectors, as a rule, consists of at least 1000 bp, which is more than 20% of the length of the vector itself. A reverse relationship between the vector length and its ability to penetrate into eukaryotic cells is observed; DNA vectors with a small length effectively penetrate into human and animal cells. For example, in a series of experiments on transfection of HeLa cells with 383-4548 bp DNA vectors it was shown that the difference in penetration efficiency can be up to two orders of magnitude (100 times different) (Hornstein B D et al.//PLoS ONE. 2016;11(12): e0167537.).

Thus, when selecting a DNA vector, for reasons of safety and maximum effectiveness, preference should be given to those constructs that do not contain antibiotic resistance genes, the sequences of viral origin and length of which allows for the effective penetration into eukaryotic cells. A strain for production of such DNA vector in quantities sufficient for the purposes of gene therapy should ensure the possibility of stable DNA vector amplification using antibiotic-free nutrient media.

Example of usage of the recombinant DNA vectors for gene therapy is the method of producing a recombinant vector for genetic immunisation (U.S. Pat. No. 9,550,998 B2. The plasmid vector is a supercoiled DNA that is used for the expression of cloned genes in human and animal cells. The vector contains an origin of replication, regulatory elements comprising human cytomegalovirus promoter and enhancer, and regulatory sequences from the human T-cell lymphotropic virus.

The vector is accumulated in a dedicated E. coli strain free of antibiotics through antisense complementation of sacB gene inserted into the strain by means of bacteriophage. The disadvantage of this invention is the presence of regulatory elements in the composition of DNA vector that constitute sequences of viral genomes.

The following applications are prototypes of this invention with regard to the use of gene therapy approaches to increase the expression level of genes from the group of SHH, CTNNB1, NOG, and WNT7A genes.

Application No. US20060105950A1 describes a method of wound healing that involves usage of gene therapy DNA or viral vectors expressing SHH. The disadvantages of this invention are vague safety requirements applied to the used vector and limited application by injury regeneration.

Application No. WO2000031134A1 describes a method for stimulating hair growth that involves usage of vectors, mainly viral, expressing CTNNB1. The disadvantage of this invention is the limited application only by stimulation of hair growth, vague safety requirements applied to the used vectors, as well as limitation of possible promoters controlling CTNNB1 expression that do not include the EF1a promoter.

U.S. Pat. No. 5,843,775A describes an invention represented by a vector expressing the NOG gene that can be potentially used to develop diagnostic tools, therapy, and biotechnology. The disadvantages of this invention are vague safety requirements applied to the used vector, as well as their ability to penetrate and express the gene in human and animal tissues.

Application No. WO2013040341A2 describes composition based on the WNT7A protein, as well as the method of use thereof for the stimulation of stem cells and in vitro, ex vivo, and in vivo tissue regeneration. One of the embodiments of this invention is the use of vectors expressing WNT7A gene, however, this application includes no specific safety requirements for the vectors used. Another disadvantage of this invention is the limited implementation of the method of use by degenerative diseases and injury regeneration.

### DISCLOSURE OF THE INVENTION

The purpose of this invention is to construct the gene therapy DNA vectors in order to increase the expression level of a group of SHH, CTNNB1, NOG (alternative name NOGG), and WNT7A genes in human and animal organisms that combine the following properties:
I) Efficiency of gene therapy DNA vector in order to increase the expression level of therapeutic genes in eukaryotic cells.
II) Possibility of safe use in gene therapy of human beings and animals due to the absence of regulatory elements representing the nucleotide sequences of viral genomes in the gene therapy DNA vector.
III) Possibility of safe use in the gene therapy of human beings and animals due to the absence of antibiotic resistance genes in the gene therapy DNA vector.
IV) Producibility and constructability of gene therapy DNA vector on an industrial scale.

Item II and III are provided for herein in line with the recommendations of the state regulators for gene therapy medicines and, specifically, the requirement of the European Medicines Agency to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies) and refrain from adding viral genomes to newly engineered plasmid vectors for gene therapy (Guideline on the quality, non-clinical and clinical aspects of gene therapy medicinal products/23 Mar. 2015, EMA/CAT/80183/2014, Committee for Advanced Therapies).

The purpose of the invention also includes the construction of strains carrying these gene therapy DNA vectors for the development and production of these gene therapy DNA vectors on an industrial scale.

The specified purpose is achieved by using the produced gene therapy DNA vector based on the gene therapy DNA vector VTvaf17 for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia, while the gene therapy DNA vector VTvaf17-SHH contains the coding region of SHH therapeutic gene cloned to gene therapy DNA vector VTvaf17 with the nucleotide sequence SEQ ID No. 1, the gene therapy DNA vector VTvaf17-CTNNB1 contains the coding region of CTNNB1 therapeutic gene cloned to gene therapy DNA vector VTvaf17 with the nucleotide sequence SEQ ID No. 2, the gene therapy DNA vector VTvaf17-NOG contains the coding region of NOG therapeutic gene cloned to gene therapy DNA vector VTvaf17 with the nucleotide sequence SEQ ID No. 3, the gene therapy DNA vector VTvaf17-WNT7A contains the coding region of WNT7A therapeutic gene cloned to gene therapy DNA vector VTvaf17 with the nucleotide sequence SEQ ID No. 4.

Each of the constructed gene therapy DNA vectors, namely VTvaf17-SHH, or VTvaf17-CTNNB1, or VTvaf17-NOG, or VTvaf17-WNT7A due to the limited size of VTvaf17 vector part not exceeding 3200 bp has the ability to efficiently penetrate into human and animal cells and express the SHH, or CTNNB1, or NOG, WNT7A therapeutic gene cloned to it.

Each of the constructed gene therapy DNA vectors, namely VTvaf17-SHH, or VTvaf17-CTNNB1, or VTvaf17-NOG, or VTvaf17-WNT7A uses nucleotide sequences that are not antibiotic resistance genes, virus genes, or regulatory elements of viral genomes as the structure elements, which ensures its safe use for gene therapy in humans and animals.

A method of gene therapy DNA vector production based on gene therapy DNA vector VTvaf17 carrying the SHH, CTNNB1, NOG, and WNT7A therapeutic gene was also developed that involves obtaining each of gene therapy DNA vectors: VTvaf17-SHH, or VTvaf17-CTNNB1, or VTvaf17-NOG, or VTvaf17-WNT7A as follows: the coding region of the SHH, or CTNNB1, or NOG, or WNT7A therapeutic gene is cloned to gene therapy DNA vector VTvaf17, and gene therapy DNA vector VTvaf17-SHH, SEQ ID No. 1, or VTvaf17-CTNNB1, SEQ ID No. 2, or VTvaf17-NOG, SEQ ID No. 3, or VTvaf17-CAT, SEQ ID No. 4, respectively, is obtained, while the coding region of the SHH, or CTNNB1, or NOG, or WNT7A therapeutic gene is obtained by isolating total RNA from the human biological tissue sample followed by the reverse transcription reaction and PCR amplification using the obtained oligonucleotides and cleaving the amplification product by corresponding restriction endonucleases, while cloning to the gene therapy DNA vector VTvaf17 is performed by BamHI and HindIII, or SalI and KpnI, or BamHI and EcoRI restriction sites, while the selection is performed without antibiotics,
at the same time, the following oligonucleotides produced for this purpose are used during gene therapy DNA vector VTvaf17-SHH, SEQ ID No. 1 production for the reverse transcription reaction and PCR amplification:

SHH_F
AGGATCCACCATGCTGCTGCTGGCGAGATGTC,
SHH_R
TATAAGCTTTCAGCTGGACTTGACCGCCAT,

and the cleaving of amplification product and cloning of the coding region of SHH gene to gene therapy DNA vector VTvaf17 is performed by BamHI and HindIII restriction endonucleases,
at the same time, the following oligonucleotides produced for this purpose are used during gene therapy DNA vector VTvaf17-CTNNB1, SEQ ID No. 2 production for the reverse transcription reaction and PCR amplification:

CTNNB1_F
ATCGTCGACCACCATGGCTACCCAAGCTGATTTG,
CTNNB1_R
TTCGGTACCTTACAGGTCAGTATCAAACCAG,

and the cleaving of amplification product and cloning of the coding region of CTNNB1 gene to gene therapy DNA vector VTvaf17 is performed by SaII and KpnI restriction endonucleases.
at the same time, the following oligonucleotides produced for this purpose are used during gene therapy DNA vector VTvaf17-NOG, SEQ ID No. 3 production for the reverse transcription reaction and PCR amplification:

NOG_F
GGATCCACCATGGAGCGCTGCCCCAG,
NOG_R
ATAGAATTCTAGCACGAGCACTTGCACT,

and the cleaving of amplification product and cloning of the coding region of NOG gene to gene therapy DNA vector VTvaf17 is performed by BamHI and EcoRI restriction endonucleases,
at the same time, the following oligonucleotides produced for this purpose are used during gene therapy DNA vector VTvaf17-WNT7A, SEQ ID No. 4 production for the reverse transcription reaction and PCR amplification:

WNT7A_F
ATCGTCGACCACCATGAACCGGAAAGCGCGGCGCT,
WNT7A_R
TTCGGTACCTCACTTGCACGTGTACATCTCCGT,

and the cleaving of amplification product and cloning of the coding region of WNT7A gene to gene therapy DNA vector VTvaf17 is performed by SaII and KpnI restriction endonucleases.

A method of use of the gene therapy DNA vector based on gene therapy DNA vector VTvaf17 carrying SHH, CTNNB1, NOG, and WNT7A therapeutic gene for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia was developed that involves transfection of the cells of patient or animal organs and tissues with the selected gene therapy DNA vector carrying the therapeutic gene based on gene therapy DNA vector VTvaf17, or several selected gene therapy DNA vectors carrying therapeutic genes based on gene therapy DNA vector VTvaf17, from the group of constructed gene therapy DNA vectors carrying therapeutic genes based on gene therapy DNA vector VTvaf17 and/or injection of autologous cells of said patient or animal transfected by the selected gene therapy DNA vector carrying therapeutic gene based on gene therapy DNA vector VTvaf17 or several selected gene therapy DNA vectors carrying the therapeutic genes based on gene therapy DNA vector VTvaf17 from the constructed gene therapy DNA vectors carrying therapeutic genes based on gene therapy DNA vector VTvaf17 into the organs and tissues of the same patient or animal and/or the injection of the selected gene therapy DNA vector carrying therapeutic gene based on gene therapy DNA vector VTvaf17 or several selected gene therapy DNA vectors carrying therapeutic genes based on gene therapy DNA vector VTvaf17 from the group of constructed gene therapy DNA vectors carrying therapeutic genes based on gene therapy DNA vector VTvaf17 into the organs and tissues of the same patient or animal, or the combination of the indicated methods.

A method of production of strain for construction of a gene therapy DNA vector for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia was developed that involves making electrocompetent cells of Escherichia coli strain SCS110-AF and subjecting these cells to electroporation with gene therapy DNA vector VTvaf17-SHH, or gene therapy DNA vector VTvaf17-CTNNB1, or gene therapy DNA vector VTvaf17-NOG, or gene therapy DNA vector VTvaf17-WNT7A. After that, the cells are poured into agar plates (Petri dishes) with a selective medium containing yeastrel, peptone, 6% sucrose, and 10 μg/ml of chloramphenicol, and as a result, Escherichia coli strain SCS110-AF/VTvaf17-SHH or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A is obtained.

Escherichia coli strain SCS110-AF/VTvaf17-SHH carrying the gene therapy DNA vector VTvaf17-SHH for production thereof allowing for antibiotic-free selection during gene therapy DNA vector production, or Escherichia coli strain SCS110-AF/VTvaf17-CTNNB1 carrying the gene therapy DNA vector VTvaf17-CTNNB1 for production thereof allowing for antibiotic-free selection during gene therapy DNA vector production, or Escherichia coli strain SCS110-AF/VTvaf17-NOG carrying the gene therapy DNA vector VTvaf17-NOG for production thereof allowing for antibiotic-free selection during gene therapy DNA vector production, or Escherichia coli strain SCS110-AF/VTvaf17-WNT7A carrying the gene therapy DNA vector VTvaf17-WNT7A for production thereof allowing for antibiotic-free selection during gene therapy DNA vector production is claimed for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia.

A method of production on an industrial scale of gene therapy DNA vector based on gene therapy DNA vector VTvaf17 carrying the SHE, or CTNNB1, or NOG, or WNT7A therapeutic gene for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia was developed that involves production of gene therapy DNA vector VTvaf17-SHH, or gene therapy DNA vector VTvaf17-CTNNB1, or gene therapy DNA vector VTvaf17-NOG, or gene therapy DNA vector VTvaf17-WNT7A by inoculating a culture flask containing the prepared medium with seed culture selected from Escherichia coli strain CS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A, then the cell culture is incubated in an incubator shaker and transferred to an industrial fermenter, then grown to a stationary phase, then the fraction containing the target DNA product is extracted, multi-stage filtered, and purified by chromatographic methods.

### BRIEF DESCRIPTION OF THE DRAWINGS 2452). The essence of the invention is explained in the drawings, where:

FIG. 1 2452). shows the structure of gene therapy DNA vector VTvaf17 carrying the therapeutic gene selected from the group of SHH, CTNNB1, NOG, and WNT7A genes that constitutes a circular double-stranded DNA molecule capable of autonomous replication in Escherichia coli cells.

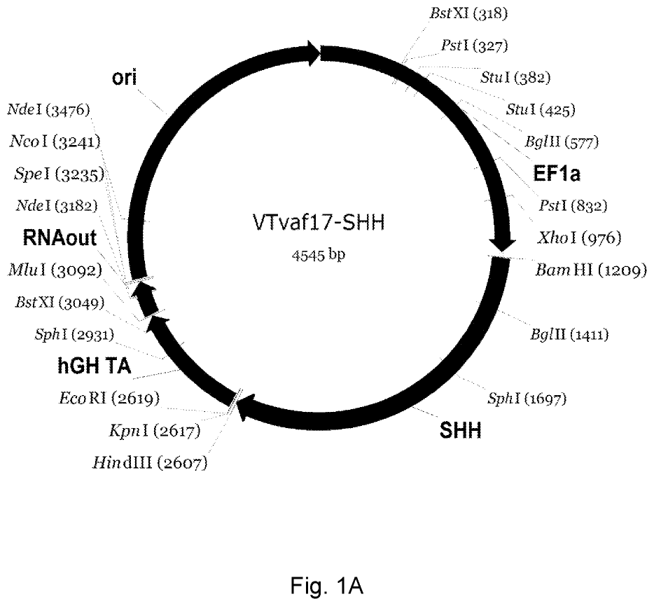

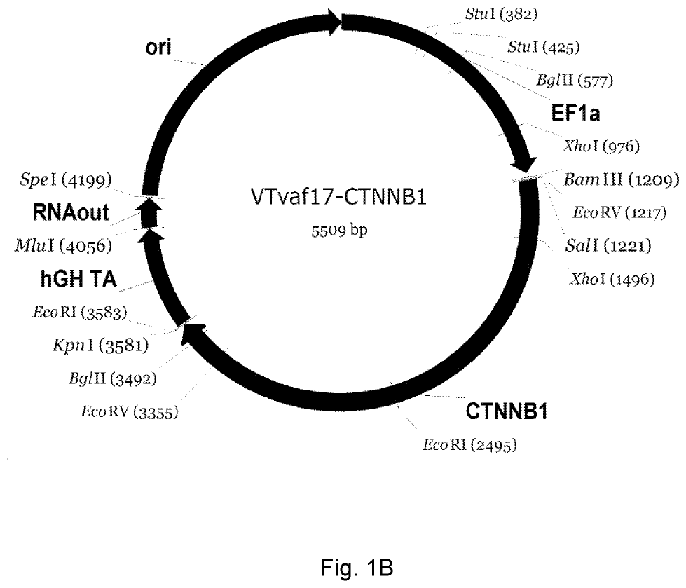

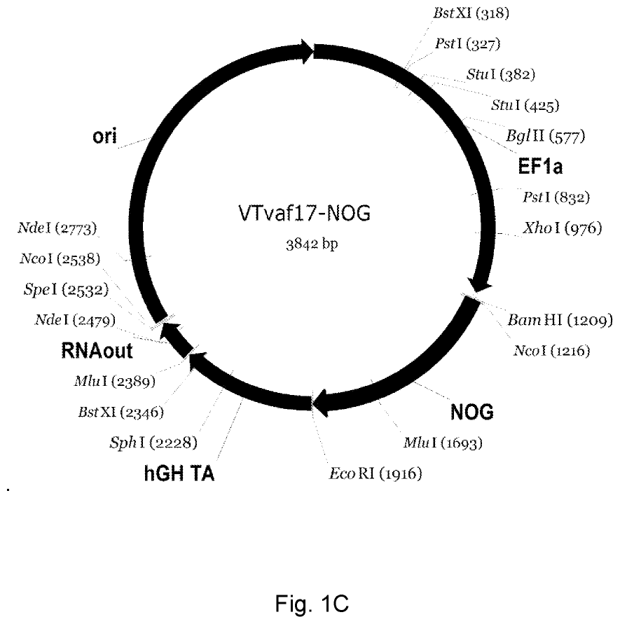

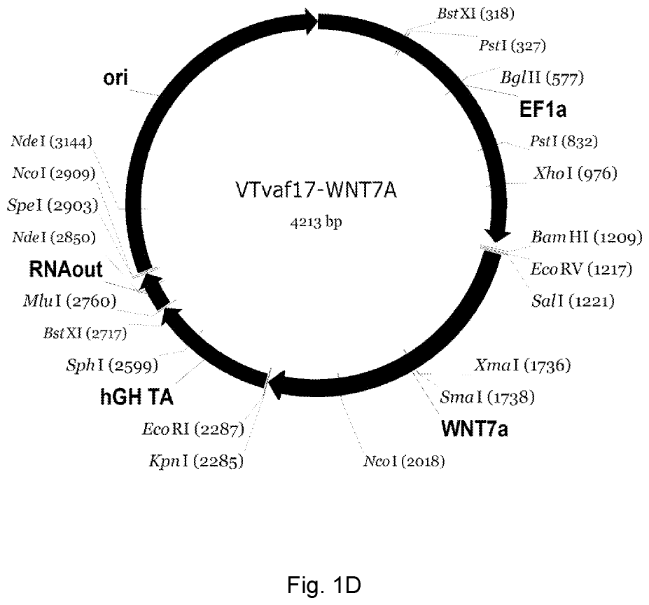

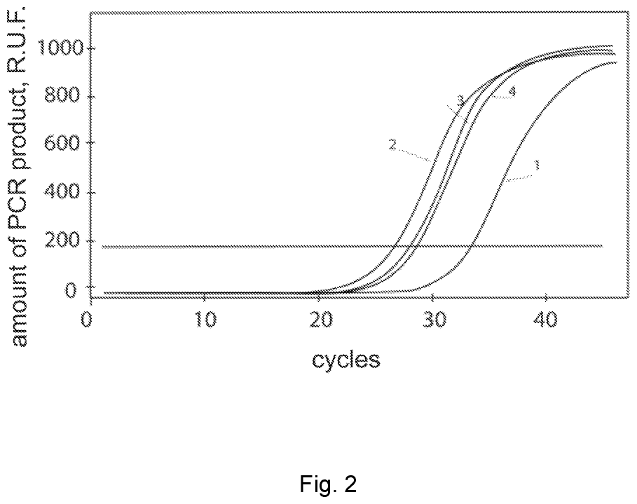

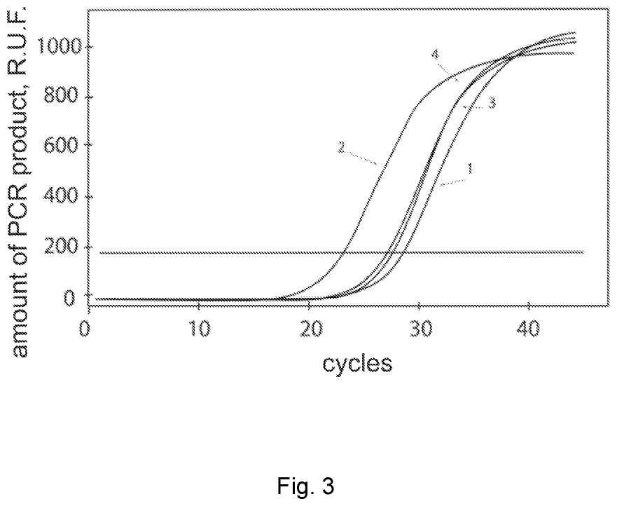

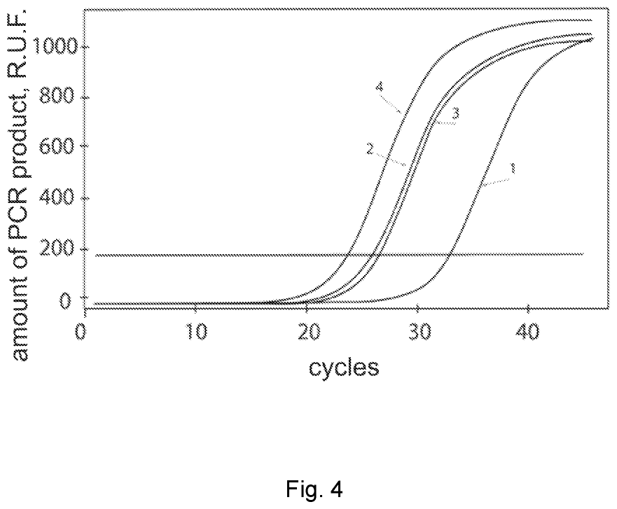

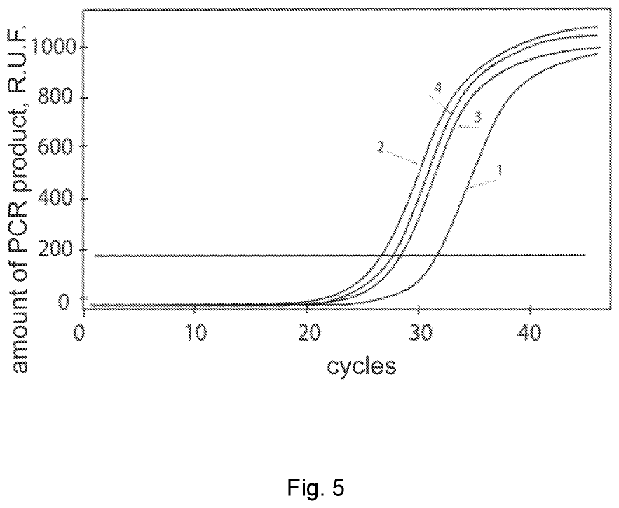

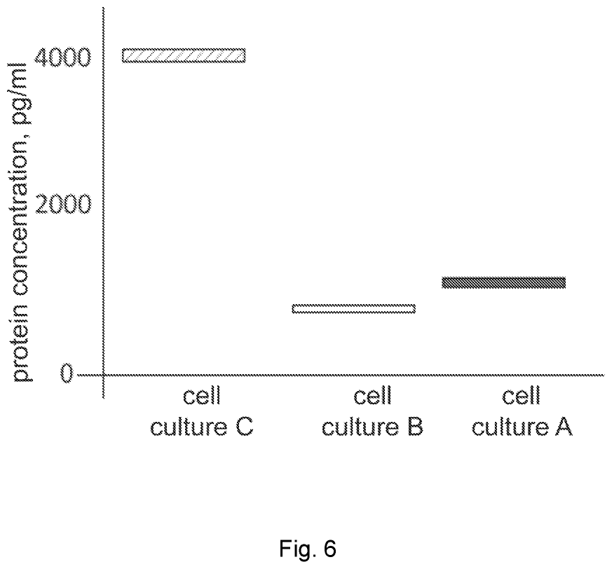

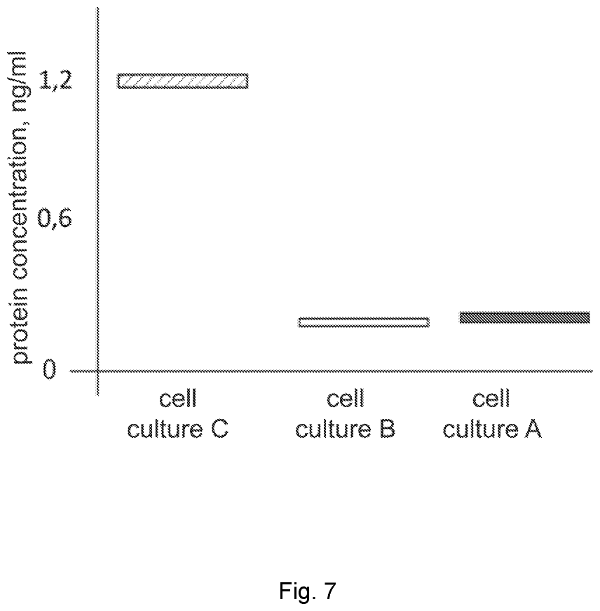

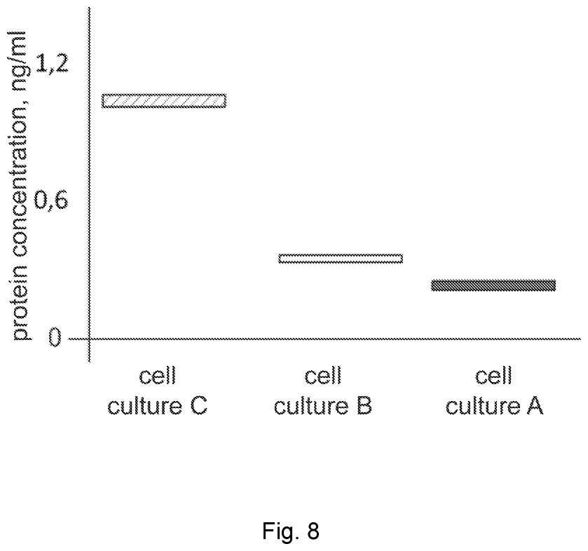

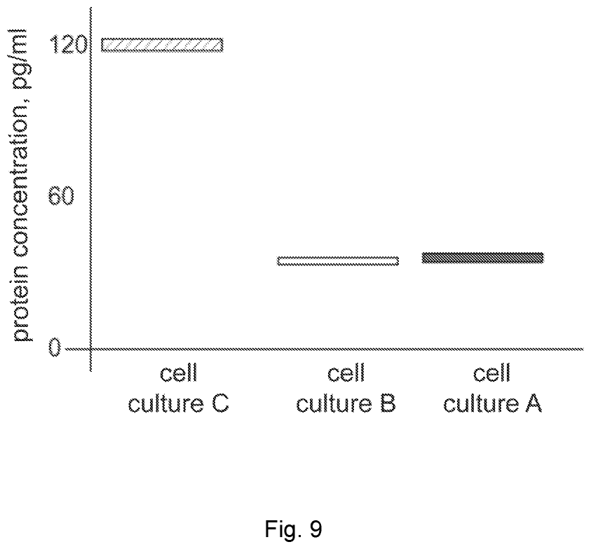

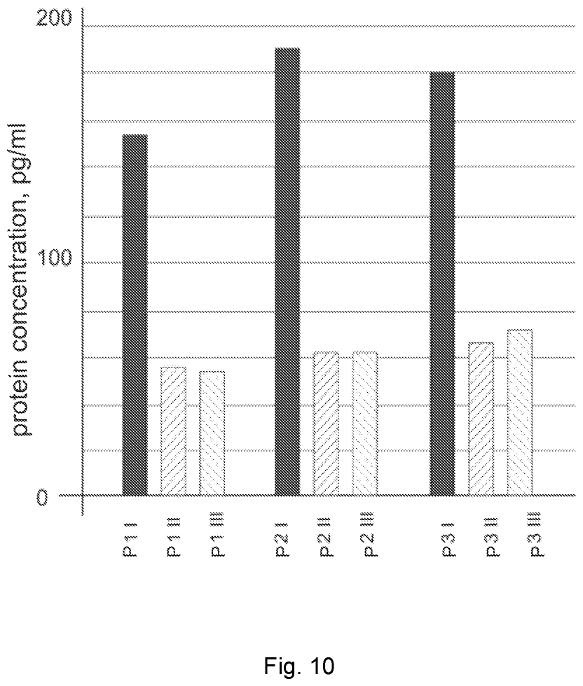

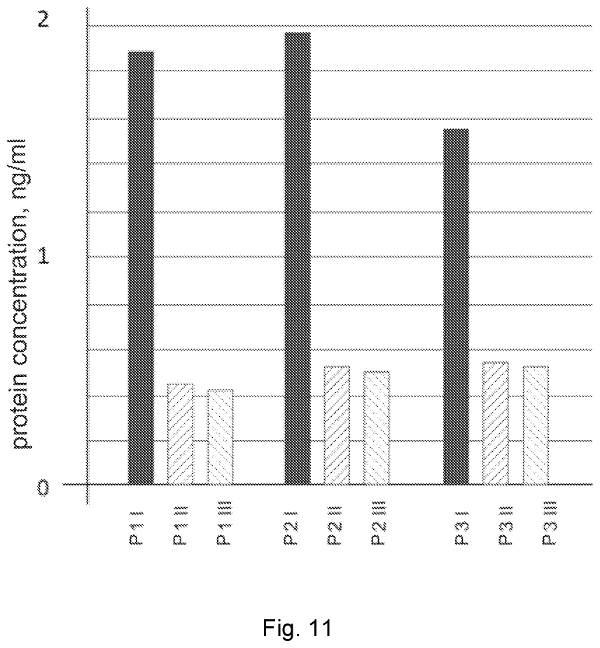

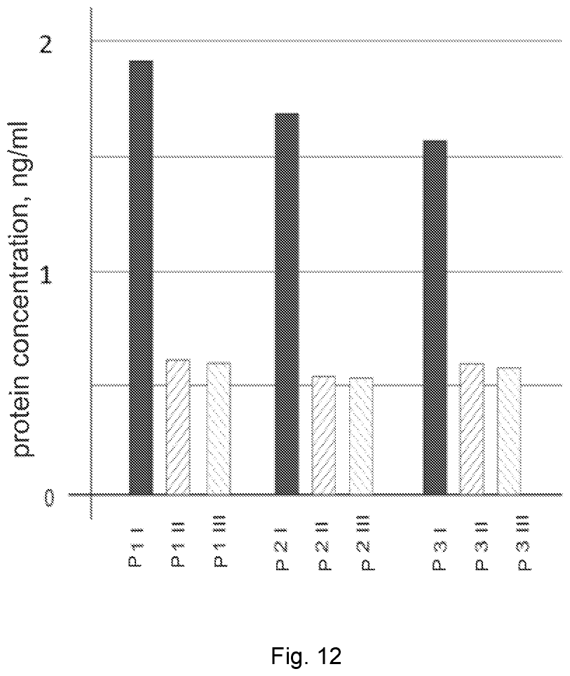

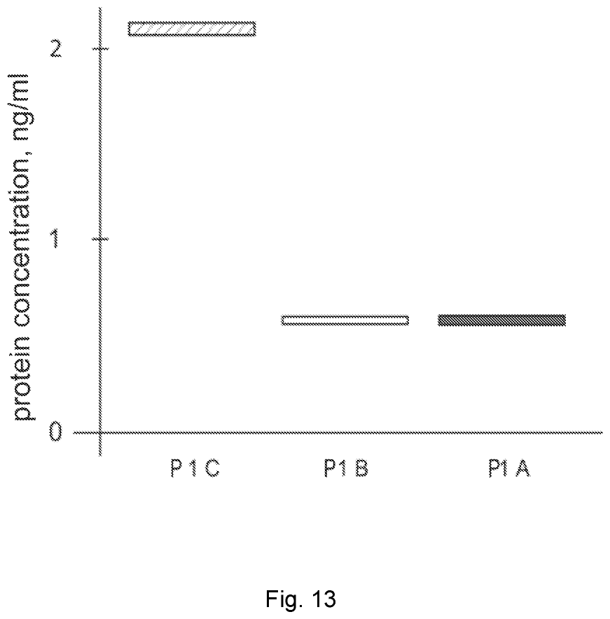

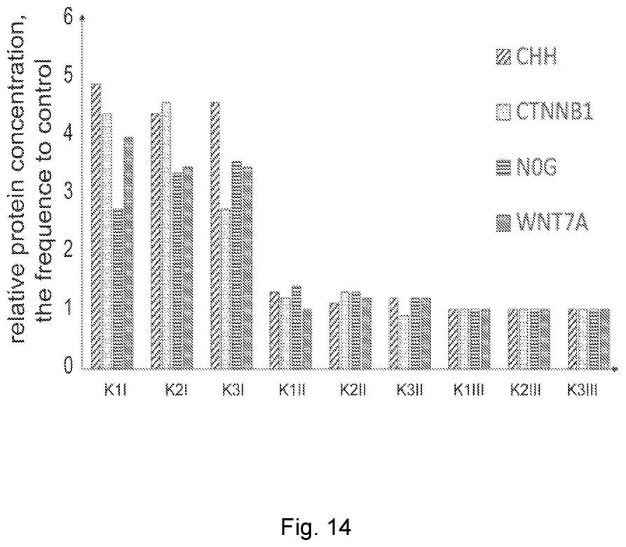

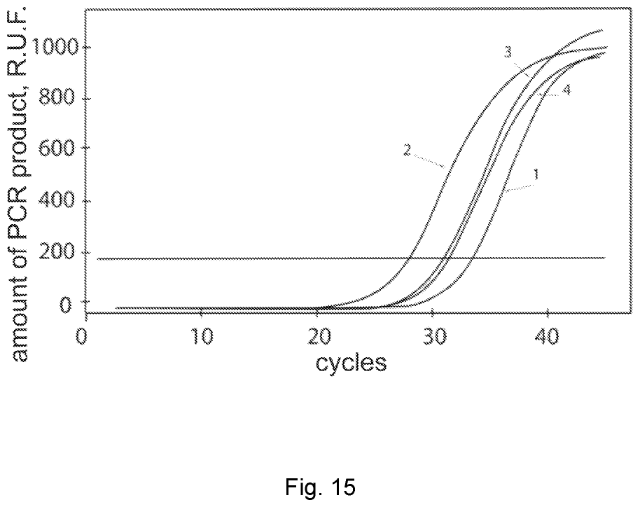

FIG. 1 shows the structures corresponding to:
A—gene therapy DNA vector VTvaf17-SHH,
B—gene therapy DNA vector VTvaf17-CTNNB1,
C—gene therapy DNA vector VTvaf17-NOG,
D—gene therapy DNA vector VTvaf17-WNT7A.

The following structural elements of the vector are indicated in the structures:

EF1a—the promoter region of human elongation factor EF1A with an intrinsic enhancer contained in the first intron of the gene. It ensures efficient transcription of the recombinant gene in most human tissues,

The reading frame of the therapeutic gene corresponding to the coding region of the SHH gene (FIG. 1A), or CTNNB1 (FIG. 1B), or NOG (FIG. 1C), or WNT7A (FIG. 1D), respectively,

hGH-TA—the transcription terminator and the polyadenylation site of the human growth factor gene,

ori—the origin of replication for autonomous replication with a single nucleotide substitution to increase plasmid production in the cells of most Escherichia coli strains,

RNA-out—the regulatory element RNA-OUT of transposon Tn 10 allowing for antibiotic-free positive selection in case of the use of Escherichia coli strain SCS 110-AF.

Unique restriction sites are marked.

FIG. 2

shows diagrams of cDNA amplicon accumulation of the therapeutic gene, namely the SHH gene, in HDFa primary human dermal fibroblast cell culture (ATCC PCS-201-012) before its transfection and 48 hours after transfection of these cells with gene therapy DNA vector VTvaf17-SHH in order to assess the ability to penetrate into eukaryotic cells and functional activity, i.e. expression of the therapeutic gene at the mRNA level.

Curves of accumulation of amplicons during the reaction are shown in FIG. 2 corresponding to:
1—cDNA of SHH gene in HDFa primary human dermal fibroblast cell culture before transfection with DNA vector VTvaf17-SHH,
2—cDNA of SHH gene in HDFa primary human dermal fibroblast cell culture after transfection with DNA vector VTvaf17-SHH,
3—cDNA of B2M gene in HDFa primary human dermal fibroblast cell culture before transfection with DNA vector VTvaf17-SHH,
4—cDNA of B2M gene in HDFa primary human dermal fibroblast cell culture after transfection with DNA vector VTvaf17-SHH.

B2M (beta-2-microglobuline) gene listed in the GenBank database under number NM 004048.2 was used as a reference gene.

FIG. 3

shows diagrams of cDNA amplicon accumulation of the therapeutic gene, namely the CTNNB1 gene, in HEKa primary human epidermal keratinocyte cell culture (ATCC PCS-200-011) before its transfection and 48 hours after transfection of these cells with gene therapy DNA vector VTvaf17-CTNNB1 in order to assess the ability to penetrate into eukaryotic cells and functional activity, i.e. expression of the therapeutic gene at the mRNA level.

Curves of accumulation of amplicons during the reaction are shown in FIG. 3 corresponding to:
1—cDNA of CTNNB1 gene in HEKa primary human epidermal keratinocyte cell culture before transfection with DNA vector VTvaf17-CTNNB1,
2—cDNA of CTNNB1 gene in HEKa primary human epidermal keratinocyte cell culture after transfection with DNA vector VTvaf17-CTNNB1,
3—cDNA of B2M gene in HEKa primary human epidermal keratinocyte cell culture before transfection with DNA vector VTvaf17-CTNNB1,
4—cDNA of B2M gene in HEKa primary human epidermal keratinocyte cell culture after transfection with DNA vector VTvaf17-CTNNB1.

B2M (beta-2-microglobuline) gene listed in the GenBank database under number NM 004048.2 was used as a reference gene.

FIG. 4

shows diagrams of cDNA amplicon accumulation of the therapeutic gene, namely the NOG gene, in HT 297.T human dermal fibroblast cell line (ATCC® CRL-7782™) before its transfection and 48 hours after transfection of these cells with gene therapy DNA vector VTvaf17-NOG in order to assess the ability to penetrate into eukaryotic cells and functional activity, i.e. expression of the therapeutic gene at the mRNA level.

Curves of accumulation of amplicons during the reaction are shown in FIG. 4 corresponding to:
1—cDNA of NOG gene in HT 297.T human dermal fibroblast cell line before transfection with DNA vector VTvaf17-NOG,
2—cDNA of NOG gene in HT 297.T human dermal fibroblast cell line after transfection with DNA vector VTvaf17-NOG,
3—cDNA of B2M gene in HT 297.T human dermal fibroblast cell line before transfection with DNA vector VTvaf17-NOG,
4—cDNA of B2M gene in HT 297.T human dermal fibroblast cell line after transfection with DNA vector VTvaf17-NOG.

B2M (beta-2-microglobuline) gene listed in the GenBank database under number NM 004048.2 was used as a reference gene.

FIG. 5

shows diagrams of cDNA amplicon accumulation of the therapeutic gene, namely the WNT7A gene, in Primary Epidermal Melanocytes; Normal, Human, Adult (HEMa) (ATCC® PCS-200-013™) before their transfection and 48 hours after transfection of these cells with the DNA vector VTvaf17-WNT7A in order to assess the ability to penetrate into eukaryotic cells and functional activity, i.e. expression of the therapeutic gene at the mRNA level.

Curves of accumulation of amplicons during the reaction are shown in FIG. 5 corresponding to:
1—cDNA of WNT7A gene in HEMa primary human epidermal melanocyte cell culture before transfection with DNA vector VTvaf17-WNT7A,
2—cDNA of WNT7A gene in HEMa primary human epidermal melanocyte cell culture after transfection with DNA vector VTvaf17-WNT7A,
3—cDNA of B2M gene in HEMa primary human epidermal melanocyte cell culture before transfection with DNA vector VTvaf17-WNT7A,
4—cDNA of B2M gene in HEMa primary human epidermal melanocyte cell culture after transfection with DNA vector VTvaf17-WNT7A.

B2M (beta-2-microglobuline) gene listed in the GenBank database under number NM 004048.2 was used as a reference gene.

FIG. 6

shows the plot of SHH protein concentration in the cell lysate of HDFa primary human dermal fibroblasts (ATCC PCS-201-01) after transfection of these cells with DNA vector VTvaf17-SHH in order to assess the functional activity, i.e. expression at the protein level based on the SHH protein concentration change in the cell lysate.

The following elements are indicated in FIG. 6:
culture A—HDFa primary human dermal fibroblast cell culture transfected with aqueous dendrimer solution without plasmid DNA (reference),
culture B—HDFa primary human dermal fibroblast cell culture transfected with DNA vector VTvaf17,
culture C—HDFa primary human dermal fibroblast cell culture transfected with DNA vector VTvaf17-SHH.

FIG. 7

shows the plot of CTNNB1 protein concentration in the lysate of HEKa primary human epidermal keratinocyte cells (ATCC PCS-200-01) after transfection of these cells with gene therapy DNA vector VTvaf17-CTNNB1 in order to assess the functional activity, i.e. the therapeutic gene expression at the protein level, and the possibility of increasing the level of protein expression by gene therapy DNA vector based on gene therapy vector VTvaf17 carrying the CTNNB1 therapeutic gene.

The following elements are indicated in FIG. 7:
culture A—HEKa primary human epidermal keratinocyte cell culture transfected with aqueous dendrimer solution without plasmid DNA (reference),
culture B—HEKa primary human epidermal keratinocyte cell culture transfected with DNA vector VTvaf17,
culture C—HEKa primary human epidermal keratinocyte cell culture transfected with DNA vector VTvaf17-CTNNB1.

FIG. 8

shows the plot of NOG protein concentration in the lysate of HT 297.T human dermal fibroblast cell line (ATCC® CRL-7782™) after transfection of these cells with DNA vector VTvaf17-NOG in order to assess the functional activity, i.e. the therapeutic gene expression at the protein level, and the possibility of increasing the level of protein expression by gene therapy DNA vector based on gene therapy vector VTvaf17 carrying the NOG therapeutic gene.

The following elements are indicated in FIG. 8:
culture A—HT 297.T human dermal fibroblast cell culture transfected with aqueous dendrimer solution without plasmid DNA (reference),
culture B—HT 297.T human dermal fibroblast cell culture transfected with DNA vector VTvaf17,
culture C—HT 297.T human dermal fibroblast cell culture transfected with DNA vector VTvaf17-NOG.

FIG. 9

shows the plot of WNT7A protein concentration in the cell lysate of Primary Epidermal Melanocytes; Normal, Human, Adult (HEMa) (ATCC® PCS-200-013™) after transfection of these cells with gene therapy DNA vector VTvaf17-WNT7A in order to assess the functional activity, i.e. the therapeutic gene expression at the protein level, and the possibility of increasing the level of protein expression by gene therapy DNA vector based on gene therapy vector VTvaf17 carrying the WNT7A therapeutic gene.

The following elements are indicated in FIG. 9:
culture A—HEMa primary epidermal melanocyte cell culture transfected with aqueous dendrimer solution without plasmid DNA (reference),
culture B—HEMa human epidermal melanocyte cell culture transfected with DNA vector VTvaf17,
culture C—HEMa human epidermal melanocyte cell culture transfected with DNA vector VTvaf17-WNT7A.

FIG. 10

shows the plot of WNT7A protein concentration in the skin biopsy samples of three patients after injection of gene therapy DNA vector VTvaf17-WNT7A into the skin of these patients in order to assess the functional activity, i.e. the expression of the therapeutic gene at the protein level, and the possibility of increasing the level of protein expression using gene therapy DNA vector based on gene therapy DNA vector VTvaf17 carrying the WNT7A therapeutic gene.

The following elements are indicated in FIG. 10:
P1I—patient P1 skin biopsy in the region of injection of gene therapy DNA vector VTvaf17-WNT7A,
P1II—patient P1 skin biopsy in the region of injection of gene therapy DNA vector VTvaf17 (placebo),
P1III—patient P1 skin biopsy from intact site,
P2I—patient P2 skin biopsy in the region of injection of gene therapy DNA vector VTvaf17-WNT7A,
P2II—patient P2 skin biopsy in the region of injection of gene therapy DNA vector VTvaf17 (placebo),
P2III—patient P2 skin biopsy from intact site,
P3I—patient P3 skin biopsy in the region of injection of gene therapy DNA vector VTvaf17-WNT7A,
P3II—patient P3 skin biopsy in the region of injection of gene therapy DNA vector VTvaf17 (placebo),
P3III—patient P3 skin biopsy from intact site.

FIG. 11

shows the plot of NOG protein concentration in the gastrocnemius muscle biopsy specimens of three patients after injection of gene therapy DNA vector VTvaf17-NOG into the gastrocnemius muscle of these patients in order to assess the functional activity, i.e. the therapeutic gene expression at the protein level, and the possibility of increasing the level of protein expression using gene therapy DNA vector based on gene therapy vector VTvaf17 carrying the NOG therapeutic gene.

The following elements are indicated in FIG. 11:
P1I—patient P1 gastrocnemius muscle biopsy in the region of injection of gene therapy DNA vector VTvaf17-NOG,
P1II—patient P1 gastrocnemius muscle biopsy in the region of injection of gene therapy DNA vector VTvaf17 (placebo),
P1III—patient P1 gastrocnemius muscle biopsy from intact site,
P2I—patient P2 gastrocnemius muscle biopsy in the region of injection of gene therapy DNA vector VTvaf17-NOG,
P2II—patient P2 gastrocnemius muscle biopsy in the region of injection of gene therapy DNA vector VTvaf17 (placebo),
P2III—patient P2 gastrocnemius muscle biopsy from intact site,
P3I—patient P3 gastrocnemius muscle biopsy in the region of injection of gene therapy DNA vector VTvaf17-NOG,
P3II—patient P3 gastrocnemius muscle biopsy in the region of injection of gene therapy DNA vector VTvaf17 (placebo),
P3III—patient P3 gastrocnemius muscle biopsy from intact site.

FIG. 12

shows the plot of CTNNB 1 protein concentration in the skin biopsy specimens of three patients after injection of gene therapy DNA vector VTvaf17-CTNNB 1 into the skin of these patients in order to assess the functional activity, i.e. the expression of the therapeutic gene at the protein level, and the possibility of increasing the level of protein expression using gene therapy DNA vector based on gene therapy vector VTvaf17 carrying the-CTNNB1 therapeutic gene.

The following elements are indicated in FIG. 12:
P1I—patient P1 skin biopsy in the region of injection of gene therapy DNA vector VTvaf17-CTNNB 1,
P1II—patient P1 skin biopsy in the region of injection of gene therapy DNA vector VTvaf17 (placebo),
P1III—patient P1 skin biopsy from intact site,
P2I—patient P2 skin biopsy in the region of injection of gene therapy DNA vector VTvaf17-CTNNB 1,
P2II—patient P2 skin biopsy in the region of injection of gene therapy DNA vector VTvaf17 (placebo),
P2III—patient P2 skin biopsy from intact site,
P3I—patient P3 skin biopsy in the region of injection of gene therapy DNA vector VTvaf17-CTNNB 1,
P3II—patient P3 skin biopsy in the region of injection of gene therapy DNA vector VTvaf17 (placebo),
P3III—patient P3 skin biopsy from intact site.

FIG. 13

shows the plot of CTNNB1 protein concentration in human skin biopsy samples after subcutaneous injection of autologous fibroblast cell culture transfected with the gene therapy DNA vector VTvaf17-CTNNB1 in order to demonstrate the method of use by injecting autologous cells transfected with the gene therapy DNA vector VTvaf17-CTNNB1.

The following elements are indicated in FIG. 13:
P1C—patient P1 skin biopsy in the region of injection of autologous fibroblast culture of the patient transfected with gene therapy DNA vector VTvaf17-CTNNB1,
P1B—patient P1 skin biopsy in the region of injection of autologous fibroblasts of the patient transfected with gene therapy DNA vector VTvaf17,
P1A—patient P1 skin biopsy from intact site.

FIG. 14

shows the plot of concentrations of human SHE protein, human CTNNB1 protein, human NOG protein, and human WNT7A protein in biopsy samples of three Wistar-Bratislava rats in the preliminary epilated area after injection into epilated area of a mixture of gene therapy vectors: gene therapy DNA vector VTvaf17-SHH, gene therapy DNA vector VTvaf17-CTNNB1, gene therapy DNA vector VTvaf17-NOG, and gene therapy DNA vector VTvaf17-WNT7A in order to demonstrate the method of use of a mixture of gene therapy DNA vectors.

The following elements are indicated in FIG. 14:
K1I—rat K1 skin biopsy sample in the region of injection of a mixture of gene therapy DNA vectors: VTvaf17-SHH, VTvaf17-CTNNB1, VTvaf17-NOG, and VTvaf17-WNT7A,
K1II—rat K1 skin biopsy sample in the region of injection of gene therapy DNA vector VTvaf17 (placebo),
K1III—rat K1 skin biopsy sample of the reference intact site,
K2I—rat K2 skin biopsy sample in the region of injection of a mixture of gene therapy DNA vectors: VTvaf17-SHH, VTvaf17-CTNNB1, VTvaf17-NOG, and VTvaf17-WNT7A,
K2II—rat K2 skin biopsy sample in the region of injection of gene therapy DNA vector VTvaf17 (placebo),
K2III—rat K2 skin biopsy sample of the reference intact site,
K3I—rat K3 skin biopsy sample in the region of injection of a mixture of gene therapy DNA vectors: VTvaf17-SHH, VTvaf17-CTNNB1, VTvaf17-NOG, and VTvaf17-WNT7A,
K3II—rat K3 skin biopsy sample in the region of injection of gene therapy DNA vector VTvaf17 (placebo),
K3III—rat K3 skin biopsy sample of the reference intact site.

FIG. 15

shows diagrams of cDNA amplicon accumulation of the NOG therapeutic gene in bovine dermal fibroblast cells (ScienCell, Cat. #B2300) before and 48 hours after transfection of these cells with the DNA vector VTvaf17-NOG in order to demonstrate the method of use by injecting the gene therapy DNA vector in animals.

Curves of accumulation of amplicons during the reaction are shown in FIG. 15 corresponding to:
1—cDNA of NOG gene in bovine dermal fibroblast cells before transfection with gene therapy DNA vector VTvaf17-NOG,
2—cDNA of NOG gene in bovine dermal fibroblast cells after transfection with gene therapy DNA vector VTvaf17-NOG,
3—cDNA of ACT gene in bovine dermal fibroblast cells before transfection with gene therapy DNA vector VTvaf17-NOG,
4—cDNA of ACT gene in bovine dermal fibroblast cells after transfection with gene therapy DNA vector VTvaf17-NOG.

Bull/cow actin gene (ACT) listed in the GenBank database under number AH001130.2 was used as a reference gene.

### EMBODIMENT OF THE INVENTION

Gene therapy DNA vectors carrying the human therapeutic genes designed to increase the expression level of these therapeutic genes in human and animal tissues were constructed based on 3165 bp DNA vector VTvaf17. The method of production of each gene therapy DNA vector carrying the therapeutic genes is to clone the protein coding sequence of the therapeutic gene selected from the group of the following genes: SHH gene (encodes SHH protein), CTNNB1 gene (encodes CTNNB1 protein), NOG gene (alternative name NOGG, encodes NOG protein (noggin)), and WNT7A gene (encodes WNT7A protein) to the polylinker of gene therapy DNA vector VTvaf17. It is known that the ability of DNA vectors to penetrate into eukaryotic cells is due mainly to the vector size. DNA vectors with the smallest size have higher penetration capability. Thus, the absence of elements in the vector that bear no functional load, but at the same time increase the vector DNA size is preferred. These features of DNA vectors were taken into account during the production of gene therapy DNA vectors based on gene therapy DNA vector VTvaf17 carrying the therapeutic gene selected from the group of SHH, CTNNB1, NOG, and WNT7A genes with no large non-functional sequences and antibiotic resistance genes in the vector, which, in addition to technological advantages and safe use, allowed for the significant reduction of size of the produced gene therapy DNA vector VTvaf17 carrying the therapeutic gene selected from the group of SHH, CTNNB1, NOG, and WNT7A genes. Thus, the ability of the obtained gene therapy DNA vector to penetrate into eukaryotic cells is due to its small length.

Each of the following gene therapy DNA vectors: VTvaf17-SHH, or VTvaf17-CTNNB1, or VTvaf17-NOG, or VTvaf17-WNT7A was produced as follows: the coding region of the therapeutic gene from the group of SHH, or CTNNB1, or NOG, or WNT7A genes was cloned to gene therapy DNA vector VTvaf17 and gene therapy DNA vector VTvaf17-SHH, SEQ ID No. 1, or VTvaf17-CTNNB1, SEQ ID No. 2, or VTvaf17-NOG, SEQ ID No. 3, or VTvaf17-WNT7A, SEQ ID No. 4, respectively, was obtained. The coding region of SHH gene (1392 bp), or CTNNB1 gene (2350 bp), or NOG gene (704 bp), or WNT7A gene (1054 bp) was produced by extracting total RNA from the biological normal tissue sample. The reverse transcription reaction was used for the synthesis of the first chain cDNA of human SHH, CTNNB1, NOG, and WNT7A genes. Amplification was performed using oligonucleotides produced for this purpose by the chemical synthesis method. The amplification product was cleaved by specific restriction endonucleases taking into account the optimal procedure for further cloning, and cloning to the gene therapy DNA vector VTvaf17 was performed by BamHI, EcoRI, and HindIII restriction sites located in the VTvaf17 vector polylinker. The selection of restriction sites was carried out in such a way that the cloned fragment entered the reading frame of expression cassette of the vector VTvaf17, while the protein coding sequence did not contain restriction sites for the selected endonucleases. Experts in this field realise that the methodological implementation of gene therapy DNA vector VTvaf17-SHH, or VTvaf17-CTNNB1, or VTvaf17-NOG, or VTvaf17-WNT7A production can vary within the framework of the selection of known methods of molecular gene cloning and these methods are included in the scope of this invention. For example, different oligonucleotide sequences can be used to amplify SHH, or CTNNB1, or NOG, or WNT7A gene, different restriction endonucleases or laboratory techniques, such as ligation independent cloning of genes.

Gene therapy DNA vector VTvaf17-SHH, or VTvaf17-CTNNB1, or VTvaf17-NOG, or VTvaf17-WNT7A has the nucleotide sequence SEQ ID No. 1, or SEQ ID No. 2, or SEQ ID No. 3, or SEQ ID No. 4, respectively. At the same time, degeneracy of genetic code is known to the experts in this field and means that the scope of this invention also includes variants of nucleotide sequences differing by insertion, deletion, or replacement of nucleotides that do not result in a change in the polypeptide sequence encoded by the therapeutic gene, and/or do not result in a loss of functional activity of the regulatory elements of VTvaf17 vector. At the same time, genetic polymorphism is known to the experts in this field and means that the scope of this invention also includes variants of nucleotide sequences of genes SHH, CTNNB1, NOG, and WNT7A genes that also encode different variants of the amino acid sequences of SHH, CTNNB1, NOG, and WNT7A proteins that do not differ from those listed in their functional activity under physiological conditions.

The ability to penetrate into eukaryotic cells and express functional activity, i.e. the ability to express the therapeutic gene of the obtained gene therapy DNA vector VTvaf17-SHH, or VTvaf17-CTNNB1, or VTvaf17-NOG, or VTvaf17-WNT7A is confirmed by introducing the obtained vector into eukaryotic cells and subsequent analysis of the expression of specific mRNA and/or protein product of the therapeutic gene. The presence of specific mRNA in cells into which the gene therapy DNA vector VTvaf17-SHH, or VTvaf17-CTNNB1, or VTvaf17-NOG, or VTvaf17-WNT7A was injected shows the ability of the obtained vector to both penetrate into eukaryotic cells and express mRNA of the therapeutic gene. Furthermore, it is known to the experts in this field that the presence of mRNA gene is a mandatory condition, but not an evidence of the translation of protein encoded by the therapeutic gene. Therefore, in order to confirm properties of the gene therapy DNA vector VTvaf17-SHH, or VTvaf17-CTNNB1, or VTvaf17-NOG, or VTvaf17-WNT7A to express the therapeutic gene at the protein level in eukaryotic cells into which the gene therapy DNA vector was injected, analysis of the concentration of proteins encoded by the therapeutic genes was carried out using immunological methods. The presence of SHH, or CTNNB1, or NOG, or WNT7A protein confirms the efficiency of expression of therapeutic genes in eukaryotic cells and the possibility of increasing the protein concentration using the gene therapy DNA vector based on gene therapy DNA vector VTvaf17 carrying the therapeutic gene selected from the group of SHH, CTNNB1, NOG, and WNT7A genes. Thus, in order to confirm the expression efficiency of the constructed gene therapy DNA vector VTvaf17-SHH carrying the therapeutic gene, namely the SHH gene, gene therapy DNA vector VTvaf17-CTNNB1 carrying the therapeutic gene, namely the CTNNB1 gene, gene therapy DNA vector VTvaf17-NOG carrying the therapeutic gene, namely the NOG gene, gene therapy DNA vector VTvaf17-WNT7A carrying the therapeutic gene, namely the WNT7A gene, the following methods were used:
A) real-time PCR, i.e. change in mRNA accumulation of therapeutic genes in human and animal cell lysate after transfection of different human and animal cell lines with gene therapy DNA vectors,
B) Enzyme-linked immunosorbent assay, i.e. change in the quantitative level of therapeutic proteins in the human cell lysate after transfection of different human cell lines with gene therapy DNA vectors,
C) Enzyme-linked immunosorbent assay, i.e. change in the quantitative level of therapeutic proteins in the supernatant of human and animals tissue biopsy specimens after the injection of gene therapy DNA vectors into these tissues,
D) Enzyme-linked immunosorbent assay, i.e. change in the quantitative level of therapeutic proteins in the supernatant of human tissue biopsies after the injection of these tissues with autologous cells of this human transfected with gene therapy DNA vectors.

In order to confirm the practicability of use of the constructed gene therapy DNA vector VTvaf17-SHH carrying the therapeutic gene, namely the SHH gene, gene therapy DNA vector VTvaf17-CTNNB1 carrying the therapeutic gene, namely the CTNNB1 gene, gene therapy DNA vector VTvaf17-NOG carrying the therapeutic gene, namely the NOG gene, gene therapy DNA vector VTvaf17-WNT7A carrying the therapeutic gene, namely the WNT7A gene, the following was performed:
A) transfection of different human and animal cell lines with gene therapy DNA vectors,
B) injection of gene therapy DNA vectors into different human and animal tissues,
C) injection of a mixture of gene therapy DNA vectors into animal tissues,
D) injection of autologous cells transfected with gene therapy DNA vectors into human tissues.

These methods of use lack potential risks for gene therapy of humans and animals due to the absence of regulatory elements in the gene therapy DNA vector that constitute the nucleotide sequences of viral genomes and absence of antibiotic resistance genes in the gene therapy DNA vector as confirmed by the lack of regions homologous to the viral genomes and antibiotic resistance genes in the nucleotide sequences of gene therapy DNA vector VTvaf17-SHH, or gene therapy DNA vector VTvaf17-CTNNB1, or gene therapy DNA vector VTvaf17-NOG, or gene therapy DNA vector VTvaf17-WNT7A (SEQ ID No. 1, or SEQ ID No. 2, or SEQ ID No. 3, or SEQ ID No. 4, respectively).

It is known to the experts in this field that antibiotic resistance genes in the gene therapy DNA vectors are used to obtain these vectors in preparative quantities by increasing bacterial biomass in a nutrient medium containing a selective antibiotic. Within the framework of this invention, in order to ensure the safe use of gene therapy DNA vector VTvaf17 carrying SHH, or CTNNB1, or NOG, or WNT7A therapeutic genes, the use of selective nutrient media containing an antibiotic is not possible. A method for obtaining strains for production of these gene therapy vectors based on Escherichia coli strain SCS110-AF is proposed as a technological solution for obtaining the gene therapy DNA vector VTvaf17 carrying a therapeutic gene selected from the group of SHH, CTNNB1, NOG, and WNT7A genes in order to scale up the production of gene therapy vectors to an industrial scale. The method of Escherichia coli strain SCS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A production involves production of competent cells of Escherichia coli strain SCS110-AF with the injection of gene therapy DNA vector VTvaf17-SHH, or DNA vector VTvaf17-CTNNB1, or DNA vector VTvaf17-NOG, or DNA vector VTvaf17-WNT7A into these cells, respectively, using transformation (electroporation) methods widely known to the specialists in this field. The obtained Escherichia coli strain SCS 110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A is used to produce the gene therapy DNA vector VTvaf17-SHH, or VTvaf17-CTNNB1, or VTvaf17-NOG, or VTvaf17-WNT7A, respectively, allowing for the use of antibiotic-free media.

In order to confirm the production of Escherichia coli strain SCS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A, transformation, selection, and subsequent tailing with extraction of plasmid DNA were performed.

To confirm the producibility and constructability and scale up of the production of gene therapy DNA vector VTvaf17-SHH carrying the therapeutic gene, namely SHH gene, gene therapy DNA vector VTvaf17-CTNNB1 carrying the therapeutic gene, namely CTNNB1 gene, gene therapy DNA vector VTvaf17-NOG carrying the therapeutic gene, namely NOG gene, gene therapy DNA vector VTvaf17-WNT7A carrying the therapeutic gene, namely WNT7A gene, to an industrial scale, the fermentation on an industrial scale of Escherichia coli strain SCS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A each containing gene therapy DNA vector VTvaf17 carrying the therapeutic gene, namely SHH, CTNNB1, NOG, and WNT7A gene was performed.

The method of scaling the production of bacterial mass to an industrial scale for the isolation of gene therapy DNA vector VTvaf17 carrying the therapeutic gene selected from the group of SHH, CTNNB1, NOG, and WNT7A genes involves incubation of the seed culture of Escherichia coli strain SCS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A in the antibiotic-free nutrient medium that provides suitable biomass accumulation dynamics. Upon reaching a sufficient amount of biomass in the logarithmic phase, the bacterial culture is transferred to an industrial fermenter and then grown to a stationary phase, then the fraction containing the therapeutic DNA product, i.e. gene therapy DNA vector VTvaf17-SHH, or gene therapy DNA vector VTvaf17-CTNNB1, or gene therapy DNA vector VTvaf17-NOG, or gene therapy DNA vector VTvaf17-WNT7A, is extracted, multi-stage filtered, and purified by chromatographic methods. It is known to the experts in this field that culture conditions of strains, composition of nutrient media (except for antibiotic-free), equipment used, and DNA purification methods may vary within the framework of standard operating procedures depending on the particular production line, but known approaches to scaling, industrial production, and purification of DNA vectors using Escherichia coli strain SCS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A fall within the scope of this invention.

The described disclosure of the invention is illustrated by examples of the embodiment of this invention.

The essence of the invention is explained in the following examples.

### EXAMPLE 1

Production of gene therapy DNA vector VTvaf17-SHH carrying the therapeutic gene, namely the SHH gene.

Gene therapy DNA vector VTvaf17-SHH was constructed by cloning the coding region of SHH gene (1392 bp) to a 3165 bp DNA vector VTvaf17 by BamHI and HindIII restriction sites. The coding region of SHH gene (1392 bp) was obtained by isolating total RNA from the biological human tissue sample followed by reverse transcription reaction using commercial kit Mint-2 (Evrogen, Russia) and PCR amplification using the following oligonucleotides:

SHH_F
AGGATCCACCATGCTGCTGCTGGCGAGATGTC,
SHH_R
TATAAGCTTTCAGCTGGACTTGACCGCCAT

and commercially available kit Phusion® High-Fidelity DNA Polymerase (New England Biolabs, USA).

Gene therapy DNA vector VTvaf17 was constructed by consolidating six fragments of DNA derived from different sources:
(a) the origin of replication was produced by PCR amplification of a region of commercially available plasmid pBR322 with a point mutation,
(b) EF1 a promoter region was produced by PCR amplification of a site of human genomic DNA,
(c) hGH-TA transcription terminator was produced by PCR amplification of a site of human genomic DNA,
(d) the RNA-OUT regulatory site of transposon Tn10 was synthesised from oligonucleotides,
(e) kanamycin resistance gene was produced by PCR amplification of a site of commercially available plasmid pET-28,
(f) the polylinker was produced by annealing two synthetic oligonucleotides.

PCR amplification was performed using the commercially available kit Phusion® High-Fidelity DNA Polymerase (New England Biolabs, USA) as per the manufacturer's instructions. The fragments have overlapping regions allowing for their consolidation with subsequent PCR amplification. Fragments (a) and (b) were consolidated using oligonucleotides Ori-F and EF1-R, and fragments (c), (d), and (e) were consolidated using oligonucleotides hGH-F and Kan-R. Afterwards, the produced fragments were consolidated by restriction with subsequent ligation by sites BamHI and NcoI. This resulted in a plasmid still devoid of the polylinker. To add it, the plasmid was cleaved by BamHI and EcoRI sites followed by ligation with fragment (f). Therefore, a vector was constructed carrying the kanamycin resistance gene flanked by SpeI restriction sites. Then this gene was cleaved by SpeI restriction sites and the remaining fragment was ligated to itself. This resulted in a 3165 bp gene therapy DNA vector VTvaf17 that is recombinant and allows for antibiotic-free selection.

The amplification product of the coding region of SHH gene and DNA vector VTvaf17 was cleaved by BamHI and HindIII restriction endonucleases (New England Biolabs, USA).

This resulted in a 4545 bp DNA vector VTvaf17-SHH with the nucleotide sequence SEQ ID No. 1 and general structure shown in FIG. 1A.

### EXAMPLE 2

Production of gene therapy DNA vector VTvaf17-CTNNB1 carrying the therapeutic gene, namely the CTNNB1 gene.

Gene therapy DNA vector VTvaf17-CTNNB1 was constructed by cloning the coding region of CTNNB1 gene (2350 bp) to a 3165 bp DNA vector VTvaf17 by SalI and KpnI restriction sites. The coding region of CTNNB1 gene (2350 bp) was obtained by isolating total RNA from the biological human tissue sample followed by reverse transcription reaction using commercial kit Mint-2 (Evrogen, Russia) and PCR amplification using the following oligonucleotides:

CTNNB1_F
ATCGTCGACCACCATGGCTACCCAAGCTGATTTG,
CTNNB1_R
TTCGGTACCTTACAGGTCAGTATCAAACCAG

and commercially available kit Phusion® High-Fidelity DNA Polymerase (New England Biolabs, USA); amplification product and DNA vector VTvaf17 were cleaved by SaII and KpnI restriction endonucleases (New England Biolabs, USA).

This resulted in a 5509 bp DNA vector VTvaf17-CTNNB1 with the nucleotide sequence SEQ ID No. 2 and general structure shown in FIG. 1B. Gene therapy DNA vector VTvaf17 was constructed as described in Example 1.

### EXAMPLE 3

Production of gene therapy DNA vector VTvaf17-NOG carrying the therapeutic gene, namely the human NOG gene.

Gene therapy DNA vector VTvaf17-NOG was constructed by cloning the coding region of NOG gene (704 bp) to a 3165 bp DNA vector VTvaf17 by BamHI and EcoRI restriction sites. The coding region of NOG gene (704 bp) was obtained by isolating total RNA from the biological human tissue sample followed by reverse transcription reaction using commercial kit Mint-2 (Evrogen, Russia) and PCR amplification using the following oligonucleotides:

NOG_F
GGATCCACCATGGAGCGCTGCCCCAG,
NOG_R
ATAGAATTCTAGCACGAGCACTTGCACT

and commercially available kit Phusion® High-Fidelity DNA Polymerase (New England Biolabs, USA); amplification product and DNA vector VTvaf17 were cleaved by restriction endonucleases BamHI and EcoRI (New England Biolabs, USA).

This resulted in a 4859 bp DNA vector VTvaf17-NOG with the nucleotide sequence SEQ ID No. 3 and general structure shown in FIG. 1C. Gene therapy DNA vector VTvaf17 was constructed as described in Example 1.

### EXAMPLE 4

Production of gene therapy DNA vector VTvaf17-WNT7A carrying the therapeutic gene, namely the WNT7A gene.

Gene therapy DNA vector VTvaf17-WNT7A was constructed by cloning the coding region of WNT7A gene (1054 bp) to a 3165 bp DNA vector VTvaf17 by BamHII and KpnI restriction sites. The coding region of WNT7A gene (105 bp) was obtained by isolating total RNA from the biological human tissue sample followed by reverse transcription reaction using commercial kit Mint-2 (Evrogen) and PCR amplification using the following oligonucleotides:

WNT7A_F
ATCGTCGACCACCATGAACCGGAAAGCGCGGCGCT,
WNT7A_R
TTCGGTACCTCACTTGCACGTGTACATCTCCGT

and commercially available kit Phusion® High-Fidelity DNA Polymerase (New England Biolabs, USA); amplification product and DNA vector VTvaf17 were cleaved by BamHII and KpnI restriction endonucleases (New England Biolabs, USA).

This resulted in a 4213 bp DNA vector VTvaf17-WNT7A with the nucleotide sequence SEQ ID No. 4 and general structure shown in FIG. 1D.

Gene therapy DNA vector VTvaf17 was constructed as described in Example 1.

### EXAMPLE 5

Proof of the ability of gene therapy DNA vector VTvaf17-SHH carrying the therapeutic gene, namely SHH gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the SHH therapeutic gene were assessed in HDFa primary human dermal fibroblast cell culture HDFa (ATCC PCS-201-01) 48 hours after its transfection with gene therapy DNA vector VTvaf17-SHH carrying the human SHH gene. The amount of mRNA was determined by the dynamics of accumulation of cDNA amplicons in the real-time PCR.

HDFa primary human dermal fibroblast cell culture was used for the assessment of changes in the therapeutic SHH mRNA accumulation. HDFa cell culture was grown under standard conditions (37° C., 5% CO2) using the Fibroblast Growth Kit—Serum-Free (ATCC® PCS-201-040). The growth medium was replaced every 48 hours during the cultivation process.

To achieve 90% confluence, 24 hours before the transfection procedure, the cells were seeded into a 24-well plate in the quantity of 5×104 cells per well. Transfection with gene therapy DNA vector VTvaf17-SHH expressing the human SHH gene was performed using Lipofectamine 3000 (ThermoFisher Scientific, USA) according to the manufacturer's recommendations. In test tube 1, 1 μl of DNA vector VTvaf17-SHH solution (concentration 500 ng/μl) and 1 μl of reagent P3000 was added to 25 μl of medium Opti-MEM (Gibco, USA). The preparation was mixed by gentle shaking. In test tube 2, 1 μl of Lipofectamine 3000 solution was added to 25 μl of medium Opti-MEM (Gibco, USA). The preparation was mixed by gentle shaking. The contents from test tube 1 were added to the contents of test tube 2, and the mixture was incubated at room temperature for 5 minutes. The resulting solution was added dropwise to the cells in the volume of 40 μl.

HDFa cells transfected with the gene therapy DNA vector VTvaf17 devoid of the inserted therapeutic gene (cDNA of SHH gene before and after transfection with gene therapy DNA vector VTvaf17 devoid of the inserted therapeutic gene is not shown in the figures) were used as a reference. Reference vector VTvaf17 for transfection was prepared as described above.

Total RNA from HDFa cells was extracted using Trizol Reagent (Invitrogen, USA) according to the manufacturer's recommendations. 1 ml of Trizol Reagent was added to the well with cells and homogenised and heated for 5 minutes at 65° C. Then the sample was centrifuged at 14,000 g for 10 minutes and heated again for 10 minutes at 65° C. Then 200 μl of chloroform was added, and the mixture was gently stirred and centrifuged at 14,000 g for 10 minutes. Then the water phase was isolated and mixed with 1/10 of the volume of 3 M sodium acetate, pH 5.2, and an equal volume of isopropyl alcohol. The sample was incubated at −20° C. for 10 minutes and then centrifuged at 14,000 g for 10 minutes. The precipitated RNA were rinsed in 1 ml of 70% ethyl alcohol, air-dried and dissolved in 10 μl of RNase-free water. The level of SHH mRNA expression after transfection was determined by assessing the dynamics of the accumulation of cDNA amplicons by real-time PCR. For the production and amplification of cDNA specific for the human SHH gene, the following SHH_SF and SHH_SR oligonucleotides were used:

SHH_SF
TTATCCCCAATGTGGCCGAG,
SHH_FR
CTGAGTCATCAGCCTGTCCG

The length of amplification product is 161 bp.

Reverse transcription reaction and PCR amplification was performed using SYBR GreenQuantitect RT-PCR Kit (Qiagen, USA) for real-time PCR. The reaction was carried out in a volume of 20 μl, containing: 25 μl of QuantiTect SYBR Green RT-PCR Master Mix, 2.5 mM of magnesium chloride, 0.5 μM of each primer, and 5 μl of RNA. For the reaction, CFX96 amplifier (Bio-Rad, USA) was used under the following conditions: 1 cycle of reverse transcription at 42° C. for 30 minutes, denaturation at 98° C. for 15 minutes, followed by 40 cycles comprising denaturation at 94° C. for 15 s, annealing of primers at 60° C. for 30s and elongation at 72° C. for 30 s. B2M (beta-2-microglobuline) gene listed in the GenBank database under number NM 004048.2 was used as a reference gene. Positive control included amplicons from PCR on matrices represented by plasmids in known concentrations containing cDNA sequences of SHH and B2M genes. Negative control included deionised water. Real-time quantification of the dynamics of accumulation of cDNA amplicons of SHH and B2M genes was conducted using the Bio-Rad CFX Manager 2.1 software (Bio-Rad, USA). Diagrams resulting from the assay are shown in FIG. 2.

FIG. 2 shows that the level of specific mRNA of human SHH gene has grown massively as a result of transfection of HDFa primary human fibroblast cell culture with gene therapy DNA vector VTvaf17-SHH, which confirms the ability of the vector to penetrate eukaryotic cells and express the SHH gene at the mRNA level. The presented results also confirm the practicability of use of gene therapy DNA vector VTvaf17-SHH in order to increase the expression level of SHH gene in eukaryotic cells.

### EXAMPLE 6

Proof of the ability of gene therapy DNA vector VTvaf17-CTNNB1 carrying the therapeutic gene, namely CTNNB1 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the CTNNB1 therapeutic gene were assessed in HEKa primary human epidermal keratinocyte cell culture (ATCC PCS-200-011) 48 hours after its transfection with gene therapy DNA vector VTvaf17-CTNNB1 carrying the human CTNNB1 gene. The amount of mRNA was determined by the dynamics of accumulation of cDNA amplicons in the real-time PCR.

HEKa primary human epidermal keratinocyte cell culture was grown in Keratinocyte Growth Kit (ATCC® PCS-200-040™) under standard conditions (37° C., 5% CO2). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-CTNNB1 expressing the human CTNNB1 gene was performed according to the procedure described in Example 5. B2M (beta-2-microglobuline) gene listed in the GenBank database under number NM 004048.2 was used as a reference gene. HEKa cell culture transfected with the gene therapy DNA vector VTvaf17 devoid of the therapeutic gene (cDNA of CTNNB1 gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 5, except for oligonucleotides with sequences different from Example 5. For the amplification of cDNA specific for the human CTNNB1 gene, the following CTNNB1_SF and CTNNB1_SR oligonucleotides were used:

CTNNB_SF
ATGACTCGAGCTCAGAGGGT,
CTNNB_SR
ATTGCACGTGTGGCAAGTTC

The length of amplification product is 197 bp.

Positive control included amplicons from PCR on matrices represented by plasmids in known concentrations containing cDNA sequences of CTNNB1 and B2M genes. Negative control included deionised water. Real-time quantification of the PCR products, i.e. CTNNB1 and B2M gene cDNAs obtained by amplification, was conducted using the Bio-Rad CFX Manager 2.1 software (Bio-Rad, USA). Diagrams resulting from the assay are shown in FIG. 3.

FIG. 3 shows that the level of specific mRNA of human CTNNB1 gene has grown massively as a result of transfection of HEKa cell culture with gene therapy DNA vector VTvaf17-CTNNB1, which confirms the ability of the vector to penetrate eukaryotic cells and express the CTNNB1 gene at the mRNA level. The presented results also confirm the practicability of use of gene therapy DNA vector VTvaf17-CTNNB1 in order to increase the expression level of CTNNB1 gene in eukaryotic cells.

### EXAMPLE 7

Proof of the ability of gene therapy DNA vector VTvaf17-NOG carrying the therapeutic gene, namely NOG gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the NOG therapeutic gene were assessed in HT 297.T human dermal fibroblast cell culture (ATCC® CRL-7782™) 48 hours after its transfection with gene therapy DNA vector VTvaf17-NOG carrying the human NOG gene. The amount of mRNA was determined by the dynamics of accumulation of cDNA amplicons in the real-time PCR.

HT 297.T human dermal fibroblast cell culture was grown in Dulbecco's Modified Eagle's Medium (DMEM) (ATCC® 30-2002™) with the addition of 10% of bovine serum (ATCC® 30-2020™) under standard conditions (37° C., 5% CO2). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-NOG expressing the human NOG gene was performed according to the procedure described in Example 5. B2M (beta-2-microglobuline) gene listed in the GenBank database under number NM 004048.2 was used as a reference gene. HT 297.T cell culture transfected with the gene therapy DNA vector VTvaf17 devoid of the therapeutic gene (cDNA of NOG gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 5, except for oligonucleotides with sequences different from Example 5. For the amplification of cDNA specific for the human NOG gene, the following NOG SF and NOG SR oligonucleotides were used:

NOG_SF
GATCTGAACGAGACGCTGCT,
NOG_SR
TAGCCCTTTGATCTCGCTCG

The length of amplification product is 192 bp.

Positive control included amplicons from PCR on matrices represented by plasmids in known concentrations containing cDNA sequences of NOG and B2M genes. Negative control included deionised water. Real-time quantification of the PCR products, i.e. NOG and B2M gene cDNAs obtained by amplification, was conducted using the Bio-Rad CFX Manager 2.1 software (Bio-Rad, USA). Diagrams resulting from the assay are shown in FIG. 4.

FIG. 4 shows that the level of specific mRNA of human NOG gene has grown massively as a result of transfection of HT 297.T human dermal fibroblast cell line with gene therapy DNA vector VTvaf17-NOG, which confirms the ability of the vector to penetrate eukaryotic cells and express the NOG gene at the mRNA level. The presented results also confirm the practicability of use of gene therapy DNA vector VTvaf17-NOG in order to increase the expression level of NOG gene in eukaryotic cells.

### EXAMPLE 8

Proof of the ability of gene therapy DNA vector VTvaf17-WNT7A carrying the therapeutic gene, namely WNT7A gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the WNT7A therapeutic gene were assessed in Primary Epidermal Melanocytes; Normal, Human, Adult (HEMa) (ATCC® PCS-200-013™) 48 hours after their transfection with gene therapy DNA-vector VTvaf17-WNT7A carrying the human WNT7A gene. The amount of mRNA was determined by the dynamics of accumulation of cDNA amplicons in the real-time PCR.

HEMa primary human epidermal melanocyte cell culture was grown in Dermal Cell Basal Medium (ATCC® PCS-200-030™) with the addition of Adult Melanocyte Growth Kit (ATCC® PCS-200-042™) under standard conditions (37° C., 5% CO2). To achieve 90% confluence, 24 hours before the transfection procedure, the cells were seeded into a 24-well plate in the quantity of 5×10⁴ cells per well. Lipofectamine 3000 (ThermoFisher Scientific, USA) was used as a transfection reagent. The transfection with gene therapy DNA vector VTvaf17-WNT7A expressing the human WNT7A gene was performed according to the procedure described in Example 5. HEMa cell culture transfected with the gene therapy DNA vector VTvaf17 devoid of the therapeutic gene (cDNA of WNT7A gene before and after transfection with gene therapy DNA vector VTvaf17 devoid of the inserted therapeutic gene is not shown in the figures) was used as a reference- RNA isolation, reverse transcription reaction, and real-time PCR were performed as described in Example 5, except for oligonucleotides with sequences different from Example 5. For the amplification of cDNA specific for the human WNT7A gene, the following WNT7A_SF and WNT7A_SR oligonucleotides were used:

WNT_SF
GCGACAAAGAGAAGCAAGGC,
WNT_SR
CTCCTCCAGGATCTTTCGGC

The length of amplification product is 185 bp.

Positive control included amplicons from PCR on matrices represented by plasmids in known concentrations containing cDNA sequences of WNT7A and B2M genes. B2M (beta-2-microglobuline) gene listed in the GenBank database under number NM 004048.2 was used as a reference gene. Negative control included deionised water. Real-time quantification of the PCR products, i.e. WNT7A and B2M gene cDNAs obtained by amplification, was conducted using the Bio-Rad CFX Manager 2.1 software (Bio-Rad, USA). Diagrams resulting from the assay are shown in FIG. 5.

FIG. 5 shows that the level of specific mRNA of human WNT7A gene has grown massively as a result of transfection of HEMa human epidermal melanocyte cell culture with gene therapy DNA vector VTvaf17-WNT7A, which confirms the ability of the vector to penetrate eukaryotic cells and express the WNT7A gene at the mRNA level. The presented results also confirm the practicability of use of gene therapy DNA vector VTvaf17-WNT7A in order to increase the expression level of WNT7A gene in eukaryotic cells.

### EXAMPLE 9

Proof of the efficiency and practicability of use of gene therapy DNA vector VTvaf17-SHH carrying the SHH gene in order to increase the expression of SHH protein in mammalian cells.

The change in the SHH protein concentration in the lysate of HDFa human dermal fibroblasts (ATCC PCS-201-01) was assessed after transfection of these cells with DNA vector VTvaf17-SHH carrying the human SHH gene.

Human dermal fibroblast cell culture was grown as described in Example 5.

To achieve 90% confluence, 24 hours before the transfection procedure, the cells were seeded into a 24-well plate in the quantity of 5×104 cells per well. The 6th generation SuperFect Transfection Reagent (Qiagen, Germany) was used for transfection. The aqueous dendrimer solution without DNA vector (A) and DNA vector VTvaf17 devoid of cDNA of SHH gene (B) were used as a reference, and DNA vector VTvaf17-SHH carrying the human SHH gene was used as the transfected agent. The DNA-dendrimer complex was prepared according to the manufacturer's procedure (QIAGEN, SuperFect Transfection Reagent Handbook, 2002) with some modifications. For cell transfection in one well of a 24-well plate, the culture medium was added to 1 μg of DNA vector dissolved in TE buffer to a final volume of 60 μl, then 5 μl of SuperFect Transfection Reagent was added and gently mixed by pipetting five times. The complex was incubated at room temperature for 10-15 minutes. Then the culture medium was taken from the wells, the wells were rinsed with 1 ml of PBS buffer. 350 μl of medium containing 10 μg/ml of gentamicin was added to the resulting complex, mixed gently, and added to the cells. The cells were incubated with the complexes for 2-3 hours at 37° C. in the presence of 5% CO2.

The medium was then removed carefully, and the live cell array was rinsed with 1 ml of PBS buffer. Then, medium containing 10 μg/ml of gentamicin was added and incubated for 24-48 hours at 37° C. in the presence of 5% CO2.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The SHH protein was assayed by enzyme-linked immunosorbent assay (ELISA) using the Sonic Hedgehog/Shh N-Terminus ELISA (R&D Systems Cat DSHH00, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of SHH protein was used. The sensitivity was at least 3.92 pg/ml, measurement range—from 15.60 pg/ml to 1000 pg/ml. R-3.0.2 was used for the statistical treatment of the results and data visualization (https://www.r-project.org/). Diagrams resulting from the assay are shown in FIG. 6.

FIG. 6 shows that the transfection of HDFa human dermal fibroblast cells with gene therapy DNA vector VTvaf17-SHH results in increased SHH protein concentration compared to reference samples, which confirms the ability of the vector to penetrate eukaryotic cells and express the SHH gene at the protein level. The presented results also confirm the practicability of use of gene therapy DNA vector VTvaf17-SHH in order to increase the expression level of SHH gene in eukaryotic cells.

### EXAMPLE 10

Proof of the efficiency and practicability of use of gene therapy DNA vector VTvaf17-CTNNB1 carrying the CTNNB1 gene in order to increase the expression of CTNNB1 protein in mammalian cells.

The change in the CTNNB1 protein concentration in the conditioned medium of the cell lysate of HEKa primary human epidermal keratinocyte cell culture (ATCC PCS-200-01) was assessed after transfection of these cells with the DNA vector VTvaf17-CTNNB1 carrying the human CTNNB1 gene. Cells were grown as described in Example 6.

The 6th generation SuperFect Transfection Reagent (Qiagen, Germany) was used for transfection. The aqueous dendrimer solution without DNA vector (A) and DNA vector VTvaf17 devoid of cDNA of CTNNB1 gene (B) were used as a reference, and DNA vector VTvaf17-CTNNB1 carrying the human CTNNB1 gene (C) was used as the transfected agent. Preparation of the DNA dendrimer complex and transfection of HEKa cells were performed according to the procedure described in Example 9.

After transfection, 0.1 ml of 1 N HCl were added to 0.5 ml of the culture broth, mixed thoroughly, and incubated for 10 minutes at room temperature. Then, the mixture was neutralised by adding 0.1 ml of 1.2 M NaOH/0.5 M HEPES (pH 7-7.6) and stirred thoroughly. Supernatant was collected and used to assay the therapeutic protein. The CTNNB1 protein was assayed by enzyme-linked immunosorbent assay (ELISA) using the Human CTNNB1/Beta Catenin ELISA Kit (Sandwich ELISA) (LifeSpan BioSciences Cat. LS-F4396-1, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of CTNNB1 protein was used. The sensitivity was at least 5.6 pg/ml, measurement range—from 15.63 pg/ml to 1000 pg/ml. R-3.0.2 was used for the statistical treatment of the results and data visualization (https://www.r-project.org/). Diagrams resulting from the assay are shown in FIG. 7.

FIG. 7 shows that the transfection of HEKa primary human epidermal keratinocyte cell culture with gene therapy DNA vector VTvaf17-CTNNB1 results in increased CTNNB1 protein concentration compared to reference samples, which confirms the ability of the vector to penetrate eukaryotic cells and express the CTNNB1 gene at the protein level. The presented results also confirm the practicability of use of gene therapy DNA vector VTvaf17-CTNNB1 in order to increase the expression level of CTNNB1 gene in eukaryotic cells.

### EXAMPLE 11

Proof of the efficiency and practicability of use of gene therapy DNA vector VTvaf17-NOG carrying the NOG gene in order to increase the expression of NOG protein in mammalian cells.

Changes in the NOG protein concentration in the lysate of HT 297.T human dermal fibroblast culture (ATCC® CRL-7782™) were assessed after transfection of these cells with gene therapy DNA vector VTvaf17-NOG carrying the human NOG gene. Cells were cultured as described in Example 7.

The 6th generation SuperFect Transfection Reagent (Qiagen, Germany) was used for transfection. The aqueous dendrimer solution without DNA vector (A) and DNA vector VTvaf17 devoid of cDNA of NOG gene (B) were used as a reference, and DNA vector VTvaf17-NOG carrying the human NOG gene was used as the transfected agent. Preparation of the DNA dendrimer complex and transfection of HT 297.T cells were performed according to the procedure described in Example 9.

After transfection, 0.1 ml of 1 N HCl were added to 0.ml of the culture broth, mixed thoroughly, and incubated for 10 minutes at room temperature. Then, the mixture was neutralised by adding 0.1 ml of 1.2 M NaOH/0.5M HEPES (pH 7-7.6) and stirred thoroughly. Supernatant was collected and used to assay the therapeutic protein. The NOG protein was assayed by enzyme-linked immunosorbent assay (ELISA) using the Human NOG/NOG ELISA Kit (Sandwich ELISA) (LifeSpan BioSciences Cat. LS-F24239-1, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of NOG protein was used. The sensitivity was at least 125 pg/ml, measurement range—from 125 pg/ml to 8000 pg/ml. R-3.0.2 was used for the statistical treatment of the results and data visualization (https://www.r-project.org/). Diagrams resulting from the assay are shown in FIG. 8.

FIG. 8 shows that the transfection of HT 297.T human dermal fibroblast cell line (ATCC® CRL-7782™) with gene therapy DNA vector VTvaf17-NOG results in increased NOG protein concentration compared to reference samples, which confirms the ability of the vector to penetrate eukaryotic cells and express the NOG gene at the protein level. The presented results also confirm the practicability of use of gene therapy DNA vector VTvaf17-NOG in order to increase the expression level of NOG gene in eukaryotic cells.

### EXAMPLE 12

Proof of the efficiency and practicability of use of gene therapy DNA vector VTvaf17-WNT7A carrying the WNT7A gene in order to increase the expression of WNT7A protein in mammalian cells.

The change in the WNT7A protein concentration in the cell lysate of Primary Epidermal Melanocytes; Normal, Human, Adult (HEMa) (ATCC® PCS-200-013™) was assessed 48 hours after its transfection with gene therapy DNA-vector VTvaf17-WNT7A carrying the human WNT7A gene. Cells were cultured as described in Example 8.

The 6th generation SuperFect Transfection Reagent (Qiagen, Germany) was used for transfection. The aqueous dendrimer solution without DNA vector (A) and DNA vector VTvaf17 devoid of cDNA of WNT7A gene (B) were used as a reference, and DNA vectorVTvaf17-WNT7A carrying the human WNT7A gene was used as the transfected agent. Preparation of the DNA dendrimer complex and transfection of HEMa cells were performed according to the procedure described in Example 9.

After transfection, 0.1 ml of 1 N HCl were added to 0.5 ml of the culture broth, mixed thoroughly, and incubated for 10 minutes at room temperature. Then, the mixture was neutralised by adding 0.1 ml of 1.2 M NaOH/0.5M HEPES (pH 7-7.6) and stirred thoroughly. Supernatant was collected and used to assay the therapeutic protein.

The WNT7A protein was assayed by enzyme-linked immunosorbent assay (ELISA) using the Human WNT7A ELISA Kit (Sandwich ELISA) (LifeSpan BioSciences Cat. LS-F7014-1, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of WNT7A protein was used. The sensitivity was 31.25 pg/ml, measurement range—from 31.25 pg/ml to 2000 pg/ml. R-3.0.2 was used for the statistical treatment of the results and data visualization (https://www.r-project.org/). Diagrams resulting from the assay are shown in FIG. 9.

FIG. 9 shows that the transfection of HEMa human dermal microvascular endothelial cell line with gene therapy DNA vector VTvaf17-WNT7A results in increased WNT7A protein concentration compared to reference samples, which confirms the ability of the vector to penetrate eukaryotic cells and express the WNT7A gene at the protein level. The presented results also confirm the practicability of use of gene therapy DNA vector VTvaf17-WNT7A in order to increase the expression level of WNT7A gene in eukaryotic cells.

### EXAMPLE 13

Proof of the efficiency and practicability of use of gene therapy DNA vector VTvaf17-WNT7A carrying the WNT7A gene in order to increase the expression of WNT7A protein in human tissues.

To prove the efficiency of gene therapy DNA vector VTvaf17-WNT7A carrying the therapeutic gene, namely the WNT7A gene, and practicability of its use, changes in WNT7A protein concentration in human skin upon injection of gene therapy DNA vector VTvaf17-WNT7A carrying the human WNT7A gene were assessed.

To analyse changes in the WNT7A protein concentration, gene therapy DNA vector VTvaf17-WNT7A carrying the WNT7A gene was injected into the forearm skin of three patients with concurrent injection of a placebo being gene therapy DNA vector VTvaf17 devoid of cDNA of WNT7A gene.

Patient 1, man, 60 y.o. (P1); Patient 2, woman, 66 y.o. (P2); Patient 3, man, 53 y.o. (P3). Polyethyleneimine Transfection reagent cG1VIP grade in-vivo-jetPEI (Polyplus Transfection, France) was used as a transport system. Gene therapy DNA vector VTvaf17-WNT7A containing cDNA of WNT7A gene and gene therapy DNA vector VTvaf17 used as a placebo not containing cDNA of WNT7A gene were dissolved in sterile nuclease-free water. To obtain a gene construct, DNA-cGMP grade in-vivo-jetPEI complexes were prepared according to the manufacturer recommendations.

Gene therapy DNA vector VTvaf17 (placebo) and gene therapy DNA vector VTvaf17-WNT7A carrying the WNT7A gene were injected in the quantity of 1 mg for each genetic construct using the tunnel method with a 30G needle to the depth of 3 mm. The injectate volume of gene therapy DNA vector VTvaf17 (placebo) and gene therapy DNA vector VTvaf17-WNT7A carrying the WNT7A gene was 0.3 ml for each genetic construct. The points of injection of each genetic construct were located at 8 to 10 cm intervals at the forearm site.

The biopsy samples were taken on the 2nd day after the injection of the genetic constructs of gene therapy DNA vectors. The biopsy samples were taken from the patients' skin in the site of injection of gene therapy DNA vector VTvaf17-WNT7A carrying the WNT7A gene (I), gene therapy DNA vector VTvaf17 (placebo) (II), and from intact skin (III) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used in order to assay the therapeutic protein by enzyme-linked immunosorbent assay (ELISA). The WNT7A protein was assayed by enzyme-linked immunosorbent assay (ELISA) using the Human WNT7A ELISA Kit (Sandwich ELISA) (LifeSpan BioSciences Cat. LS-F7014-1, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of WNT7A protein was used. The sensitivity was 31.25 pg/ml, measurement range—from 31.25 pg/ml to 2000 pg/ml. R-3.0.2 was used for the statistical treatment of the results and data visualization (https://www.r-project.org/) according to the manufacturer's method with optical density detection using ChemWell Automated EIA and Chemistry Analyser (Awareness Technology Inc., USA). Diagrams resulting from the assay are shown in FIG. 10.

FIG. 10 shows the increased WNT7A protein concentration in the skin of all three patients in the injection site of gene therapy DNA vector VTvaf17-WNT7A carrying the human WNT7A therapeutic gene compared to the WNT7A protein concentration in the injection site of gene therapy DNA vector VTvaf17 (placebo) devoid of the human WNT7A gene, which indicates the efficiency of gene therapy DNA vector VTvaf17-WNT7A and confirms the practicability of its use, in particular upon injection of gene therapy DNA vector in human tissues.

### EXAMPLE 14

Proof of the efficiency and practicability of use of gene therapy DNA vector VTvaf17-NOG carrying the NOG gene in order to increase the expression of NOG protein in human tissues.

To prove the efficiency of gene therapy DNA vector VTvaf17-NOG carrying the NOG therapeutic gene and practicability of its use, the change in the NOG protein concentration in human muscle tissues upon injection of gene therapy DNA vector VTvaf17-NOG carrying the therapeutic gene, namely the human NOG gene, was assessed.

To analyse changes in the concentration of NOG protein, gene therapy DNA vector VTvaf17-NOG carrying the NOG gene with transport molecule was injected into the skin of three patients with concurrent injection of a placebo being gene therapy DNA vector VTvaf17 devoid of cDNA of NOG gene with transport molecule.

Patient 1, woman, 49 y.o. (P1); Patient 2, man, 53 y.o. (P2); Patient 3, man, 64 y.o. (P3). Polyethyleneimine Transfection reagent cGMP grade in-vivo-jetPEI (Polyplus Transfection, France) was used as a transport system; sample preparation was carried out in accordance with the manufacturer's recommendations.

Gene therapy DNA vector VTvaf17 (placebo) and gene therapy DNA vector VTvaf17-NOG carrying the NOG gene were injected in the quantity of 1 mg for each genetic construct using the tunnel method with a 30G needle to the depth of around 10 mm. The injectate volume of gene therapy DNA vector VTvaf17 (placebo) and gene therapy DNA vector VTvaf17-NOG carrying the NOG gene was 0.3 ml for each genetic construct. The points of injection of each genetic construct were located medially at 8 to 10 cm intervals.

The biopsy samples were taken on the 2nd day after the injection of the genetic constructs of gene therapy DNA vectors. The biopsy samples were taken from the patients' muscle tissues in the site of injection of gene therapy DNA vector VTvaf17-NOG carrying the NOG gene (I), gene therapy DNA vector VTvaf17 (placebo) (II), and intact site of gastrocnemius muscle (III) using the skin biopsy device MAGNUM (BARD, USA). The skin of patients in the biopsy site was preliminarily rinsed with sterile saline and anaesthetised with a lidocaine solution. The biopsy sample size was ca. 20 mm3, and the weight was up to 22 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein.

The NOG protein was assayed by enzyme-linked immunosorbent assay (ELISA) using the Human NOG/NOG ELISA Kit (Sandwich ELISA) (LifeSpan BioSciences Cat. LS-F24239-1, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of NOG protein was used. The sensitivity was at least 125 pg/ml, measurement range—from 125 pg/ml to 8000 pg/ml. R-3.0.2 was used for the statistical treatment of the results and data visualisation (https://www.r-project.org/). Diagrams resulting from the assay are shown in FIG. 11.

FIG. 11 shows the increased NOG protein concentration in the gastrocnemius muscle of all three patients in the injection site of gene therapy DNA vector VTvaf17-NOG carrying the therapeutic gene, namely NOG gene, compared to the NOG protein concentration in the injection site of gene therapy DNA vector VTvaf17 (placebo) devoid of the human NOG gene, which indicates the efficiency of gene therapy DNA vector VTvaf17-NOG and confirms the practicability of its use, in particular upon intramuscular injection of gene therapy DNA vector in human tissues.

### EXAMPLE 15

Proof of the efficiency and practicability of use of gene therapy DNA vector VTvaf17-CTNNB1 carrying the CTNNB1 gene in order to increase the expression of CTNNB1 protein in human tissues.

To prove the efficiency of gene therapy DNA vector VTvaf17-CTNNB1 carrying the therapeutic gene, namely the CTNNB1 gene, and practicability of its use, changes in CTNNB1 protein concentration in human skin upon injection of gene therapy DNA vector VTvaf17-CTNNB1 carrying the human CTNNB1 gene were assessed.

To analyse changes in the CTNNB1 protein concentration, gene therapy DNA vector VTvaf17-CTNNB1 carrying the CTNNB1 gene was injected into the forearm skin of three patients with concurrent injection of a placebo being gene therapy DNA vector VTvaf17 devoid of cDNA of CTNNB1 gene.

Patient 1, woman, 57 y.o. (P1); Patient 2, man, 50 y.o. (P2); Patient 3, man, 59 y.o. (P3). Polyethyleneimine Transfection reagent cGMP grade in-vivo-jetPEI (Polyplus Transfection, France) was used as a transport system. Gene therapy DNA vector VTvaf17-CTNNB1 containing cDNA of CTNNB1 gene and gene therapy DNA vector VTvaf17 used as a placebo not containing cDNA of CTNNB1 gene were dissolved in sterile nuclease-free water. To obtain a gene construct, DNA-cGMP grade in-vivo-jetPEI complexes were prepared according to the manufacturer recommendations.

Gene therapy DNA vector VTvaf17 (placebo) and gene therapy DNA vector VTvaf17-CTNNB1 carrying the CTNNB1 gene were injected in the quantity of 1 mg for each genetic construct using the tunnel method with a 30G needle to the depth of 3 mm. The injectate volume of gene therapy DNA vector VTvaf17 (placebo) and gene therapy DNA vector VTvaf17-CTNNB1 carrying the CTNNB1 gene was 0.3 ml for each genetic construct. The points of injection of each genetic construct were located at 8 to 10 cm intervals at the forearm site.

The biopsy samples were taken on the 2nd day after the injection of the genetic constructs of gene therapy DNA vectors. The biopsy samples were taken from the patients' skin in the site of injection of gene therapy DNA vector VTvaf17-CTNNB1 carrying the CTNNB1 gene (I), gene therapy DNA vector VTvaf17 (placebo) (II), and from intact skin (III) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used in order to assay the therapeutic protein by enzyme-linked immunosorbent assay (ELISA) using Human CTNNB1/Beta Catenin ELISA Kit (Sandwich ELISA) (LifeSpan BioSciences Cat. LS-F4396-1, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of CTNNB1 protein was used. The sensitivity was at least 5.6 pg/ml, measurement range—from 15.63 pg/ml to 1000 pg/ml. R-3.0.2 was used for the statistical treatment of the results and data visualization (https://www.r-project.org/). Diagrams resulting from the assay are shown in FIG. 12.

FIG. 12 shows the increased CTNNB1 protein concentration in the skin of all three patients in the injection site of gene therapy DNA vector VTvaf17-CTNNB1 carrying the human CTNNB1 therapeutic gene compared to the CTNNB1 protein concentration in the injection site of gene therapy DNA vector VTvaf17 (placebo) devoid of the human CTNNB1 gene, which indicates the efficiency of gene therapy DNA vector VTvaf17-CTNNB1 and confirms the practicability of its use, in particular upon intracutaneous injection of gene therapy DNA vector in human tissues.

### EXAMPLE 16

Proof of the efficiency of gene therapy DNA vector VTvaf17-CTNNB1 carrying the CTNNB1 gene and practicability of its use in order to increase the expression level of the CTNNB1 protein in human tissues by introducing autologous fibroblasts transfected with gene therapy DNA vector VTvaf17-CTNNB1.

To prove the efficiency of gene therapy DNA vector VTvaf17-CTNNB1 carrying the CTNNB1 gene and practicability of its use, changes in the CTNNB1 protein level in patient's skin upon injection of autologous fibroblast culture of the same patient transfected with gene therapy DNA vector VTvaf17-CTNNB1 were assessed.

The appropriate autologous fibroblast culture transfected with the gene therapy DNA vector VTvaf17-CTNNB1 carrying the CTNNB1 gene was injected into the patient's forearm skin with concurrent injection of a placebo in the form of autologous fibroblast culture transfected with gene therapy DNA vector VTvaf17 not carrying the CTNNB1 gene.

The human primary fibroblast culture was isolated from the patient skin biopsy specimens. Biopsy specimens of the skin from the area protected by ultraviolet, namely behind the ear or on the inner lateral side of the elbow, were taken using the skin biopsy device Epitheasy 3.5 (Medax SRL, Italy). The biopsy sample was ca. 10 mm and ca. 11 mg. The patient's skin was preliminarily rinsed with sterile saline and anaesthetised with a lidocaine solution. The primary cell culture was cultivated at 37° C. in the presence of 5% CO2, in the DMEM medium with 10% fetal bovine serum and 100 U/ml of ampicillin. The passage and change of culture medium were performed every 2 days. Total duration of culture growth did not exceed 25-30 days. Then an aliquot of 5×104 cells was taken from the cell culture. The patient's fibroblast culture was transfected with the gene therapy DNA vector VTvaf17-CTNNB1 carrying the CTNNB1 gene or placebo, i.e. vector VTvaf17 not carrying the CTNNB1 therapeutic gene.

The transfection was carried out using a cationic polymer such as polyethyleneimine JETPEI (Polyplus transfection, France), according to the manufacturer's instructions. The cells were cultured for 72 hours and then injected into the patient. Injection of autologous fibroblast culture of the patient transfected with gene therapy DNA vector VTvaf17-CTNNB1, and autologous fibroblast culture of the patient non-transfected with gene therapy DNA vector VTvaf17 as a placebo was performed in the forearm using the tunnel method with a 13 mm long 30G needle to the depth of approximately 3 mm. The concentration of the modified autologous fibroblasts in the introduced suspension was approximately 5 mln cells per 1 ml of the suspension, the dose of the injected cells did not exceed 15 mln. The points of injection of the autologous fibroblast culture were located at 8 to 10 cm intervals.

Biopsy specimens were taken on the 4th day after the injection of autologous fibroblast culture transfected with the gene therapy DNA vector VTvaf17-CTNNB1 carrying the therapeutic gene, namely CTNNB1 gene, and placebo. Biopsy was taken from the patient's skin in the site of injection of autologous fibroblast culture transfected with gene therapy DNA vector VTvaf17-CTNNB1 carrying the therapeutic gene, namely CTNNB1 gene (C), autologous fibroblast culture transfected with gene therapy DNA vector VTvaf17 not carrying the CTNNB1 therapeutic gene (placebo) (B), as well as from intact skin site (A) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein as described in Example 15.

Diagrams resulting from the assay are shown in FIG. 13.

FIG. 13 shows the increased concentration of CTNNB1 protein in the area of the patient's skin in the injection site of autologous fibroblast culture transfected with the gene therapy DNA vector VTvaf17-CTNNB1 carrying the CTNNB1 gene compared to the CTNNB1 protein concentration in the injection site of autologous fibroblast culture transfected with the gene therapy DNA vector VTvaf17 not carrying the CTNNB1 gene (placebo), which indicates the efficiency of gene therapy DNA vector VTvaf17-CTNNB1 and practicability of its use in order to increase the expression level of CTNNB1 in human organs, in particular upon injection of autologous fibroblasts transfected with the gene therapy DNA vector VTvaf17-CTNNB1 into the skin.

### EXAMPLE 17

Proof of the efficiency and practicability of combined use of gene therapy DNA vector VTvaf17-SHH carrying the SHH gene, gene therapy DNA vector VTvaf17-CTNNB1 carrying the CTNNB1 gene, gene therapy DNA vector VTvaf17-NOG carrying the NOG gene, and gene therapy DNA vector VTvaf17-WNT7A carrying the WNT7A gene for the upregulation of expression level of SHH, CTNNB1, NOG, and WNT7A proteins in mammalian tissues.

The change in the SHH, CTNNB1, NOG, and WNT7A protein concentration in the site of preliminary epilated rat skin was assessed when a mixture of gene therapy vectors was injected into this site.

Epilation in a group of 3 Wistar rats was performed under general anesthesia on a 2×4 cm site in accordance with known technique (Li H et al.//Sci Rep. 2017 Aug. 4;7(1):7272).

Polyethyleneimine Transfection reagent cGMP grade in-vivo-jetPEI (Polyplus Transfection, France) was used as a transport system. Equimolar mixture of gene therapy DNA vectors was dissolved in sterile nuclease-free water. To obtain a gene construct, DNA-cGMP grade in-vivo-jetPEI complexes were prepared according to the manufacturer recommendations. The injectate volume was 0.3 ml with a total quantity of DNA of 100 m. The solution was injected by tunnel method with a 30G needle to the depth of 2-3 mm in the site of preliminary epilated rat skin 48 hours after the procedure.

The biopsy samples were taken on the 2nd day after the injection of the gene therapy DNA vectors. The biopsy sample was taken from the scar areas on the skin of animals in the injection site of a mixture of four gene therapy DNA vectors carrying the genes SHH, CTNNB1, NOG, and WNT7A (site I), gene therapy DNA vector VTvaf17 (placebo) (site II), as well as from the similar skin site, not subjected to any manipulations (site III), using the skin biopsy device Epitheasy 3.5 (Medax SRL). The biopsy sample site was preliminarily rinsed with sterile saline and anaesthetised with a lidocaine solution. The biopsy sample size was ca. 10 mm3, and the weight was approximately 11 mg. Each sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic proteins as described in Example 9 (quantification of SHH protein), Example 10 (quantification of CTNNB1 protein), Example 11 (quantification of NOG protein), and Example 12 (quantification of WNT7A protein). Diagrams resulting from the assay are shown in FIG. 14.

FIGS. 14A and 14B demonstrate that the concentration of SHH, CTNNB1, NOG, and WNT7A proteins was increased in the site of preliminary epilated skin of rats (site I) where a mixture of gene therapy DNA vector VTvaf17-SHH carrying the SHH therapeutic gene, gene therapy DNA vector VTvaf17-CTNNB1 carrying the CTNNB1 therapeutic gene, gene therapy DNA vector VTvaf17-NOG carrying the NOG therapeutic gene, gene therapy DNA vector VTvaf17-WNT7A carrying the WNT7A therapeutic gene were injected compared to site II (placebo site) and site III (intact site). The obtained results show the efficiency of combined use of gene therapy DNA vectors and practicability of use for the upregulation of the expression level of therapeutic proteins in mammalian tissues.

### EXAMPLE 18

Proof of the efficiency of gene therapy DNA vector VTvaf17-NOG carrying the NOG gene and practicability of its use in order to increase the expression level of NOG protein in mammalian cells.

To prove the efficiency of gene therapy DNA vector VTvaf17-NOG carrying the NOG gene, changes in mRNA accumulation of the NOG therapeutic gene in bovine dermal fibroblast cells (ScienCell, Cat. #B2300) 48 hours after their transfection with gene therapy DNA vector VTvaf17-NOG carrying the human NOG gene were assessed.

Bovine dermal fibroblast cells BDF (ScienCell, Cat. #B2300) were grown in the FM-2 medium (ScienCell, Cat. #2331). Transfection with gene therapy DNA vector VTvaf17-NOG carrying the human NOG gene and DNA vector VTvaf17 not carrying the human NOG gene (reference), RNA extraction, reverse transcription reaction, PCR amplification, and data analysis were performed as described in Example 7. Bull/cow actin gene (ACT) listed in the GenBank database under number AH001130.2 was used as a reference gene. Positive control included amplicons from PCR on matrices represented by plasmids in known concentrations containing NOG and ACT gene sequences. Negative control included deionised water. Real-time quantification of the PCR products, i.e. NOG and ACT gene cDNAs obtained by amplification, was conducted using the Bio-Rad CFX Manager 2.1 software (Bio-Rad, USA).

Diagrams resulting from the assay are shown in FIG. 15.

FIG. 15 shows that the level of specific mRNA of human NOG gene has grown massively as a result of transfection of bovine dermal fibroblast cells BDF with gene therapy DNA vector VTvaf17-NOG, which confirms the ability of the vector to penetrate eukaryotic cells and express the NOG gene at the mRNA level. The presented results confirm the practicability of use of gene therapy DNA vector VTvaf17-NOG in order to increase the expression level of NOG gene in mammalian cells.

### EXAMPLE 19

Escherichia coli strain SCS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A carrying gene therapy DNA vector, method of production thereof.

The construction of strain for the production of gene therapy DNA vector based on gene therapy DNA vector VTvaf17 carrying the therapeutic gene on an industrial scale selected from the group of the following genes: SHH, CTNNB1, NOG, and WNT7A, namely Escherichia coli strain SCS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A carrying the gene therapy DNA vector VTvaf17-SHH, or VTvaf17-CTNNB1, or VTvaf17-NOG, or VTvaf17-WNT7A, respectively, for its production allowing for antibiotic-free selection involves making electrocompetent cells of Escherichia coli strain SCS110-AF and subjecting these cells to electroporation with gene therapy DNA vector VTvaf17-SHH, or DNA vector VTvaf17-CTNNB1, or DNA vector VTvaf17-NOG, or DNA vector VTvaf17-WNT7A. After that, the cells were poured into agar plates (Petri dishes) with a selective medium containing yeastrel, peptone, 6% sucrose, and 10 μg/ml of chloramphenicol. At the same time, production of Escherichia coli strain SCS110-AF for the production of gene therapy DNA vector VTvaf17 or gene therapy DNA vectors based on it allowing for antibiotic-free positive selection involves constructing a 64 bp linear DNA fragment that contains regulatory element RNA-IN of transposon Tn10 allowing for antibiotic-free positive selection, a 1422 bp levansucrase gene sacB, the product of which ensures selection within a sucrose-containing medium, a 763 bp chloramphenicol resistance gene catR required for the selection of strain clones in which homologous recombination occurs, and two homologous sequences, 329 bp and 233 bp, ensuring homologous recombination in the region of gene recA concurrent with gene inactivation, and then the Escherichia coli cells are transformed by electroporation, and clones surviving in a medium containing 10 μg/ml of chloramphenicol are selected.

The obtained strains for production were included in the collection of the National Biological Resource Centre—Russian National Collection of Industrial Microorganisms (NBRC RNCIM), RF and NCIMB Patent Deposit Service, UK under the following registration numbers:
Escherichia coli strain SCS110-AF/VTvaf17-SHH—registered at the Russian National Collection of Industrial Microorganisms under number: B-13253, date of deposit: 24 Sep. 2018; accession No. NCIMB: 43211, date of deposit: 20 Sep. 2018.
Escherichia coli strain SCS110-AF/VTvaf17-CTNNB1—registered at the Russian National Collection of Industrial Microorganisms under number B-13275, date of deposit: 16 Oct. 2018; accession No. NCIMB: 43301, date of deposit: 13 Dec. 2018.
Escherichia coli strain SCS110-AF/VTvaf17-NOG—registered at the Russian National Collection of Industrial Microorganisms under number: B-13256, date of deposit: 24 Sep. 2018; accession No. NCIMB: 43208, date of deposit: 20 Sep. 2018.
Escherichia coli strain SCS110-AF/VTvaf17-WNT7A—registered at the Russian National Collection of Industrial Microorganisms under number: B-13270, date of deposit: 16 Oct. 2018, accession No. NCIMB: 43305, date of deposit: 13 Dec. 2018.

### EXAMPLE 20

The method for scaling up of the gene therapy DNA vector based on gene therapy DNA vector VTvaf17 carrying the therapeutic gene selected from the group of SHH, CTNNB1, NOG, and WNT7A genes to an industrial scale.

To confirm the producibility and constructability of gene therapy DNA vector VTvaf17-SHH (SEQ ID No. 1), or VTvaf17-CTNNB1 (SEQ ID No. 2), or VTvaf17-NOG (SEQ ID No. 3), or VTvaf17-WNT7A (SEQ ID No. 4) on an industrial scale, large-scale fermentation of Escherichia coli strain SCS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A each containing gene therapy DNA vector VTvaf17 carrying the therapeutic gene, namely SHH, or CTNNB1, or NOG, or WNT7A, was performed. Each Escherichia coli strain SCS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A was produced based on Escherichia coli strain SCS110-AF (Cell and Gene Therapy LLC, United Kingdom) as described in Example 21 by electroporation of competent cells of this strain with the gene therapy DNA vector VTvaf17-SHH, or VTvaf17-CTNNB1, or VTvaf17-NOG, or VTvaf17-WNT7A carrying the therapeutic gene, namely SHH, or CTNNB1, or NOG, or WNT7A, with further inoculation of transformed cells into agar plates (Petri dishes) with a selective medium containing yeastrel, peptone, and 6% sucrose, and selection of individual clones.

Fermentation of Escherichia coli SCS110-AF/VTvaf17-SHH carrying gene therapy DNA vector VTvaf17-SHH was performed in a 10 l fermenter with subsequent extraction of gene therapy DNA vector VTvaf17-SHH.

For the fermentation of Escherichia coli strain SCS110-AF/VTvaf17-SHH, a medium was prepared containing (per 10 l of volume): 100 g of tryptone and 50 g of yeastrel (Becton Dickinson, USA); then the medium was diluted with water to 8800 ml and autoclaved at 121° C. for 20 minutes, and then 1200 ml of 50% (w/v) sucrose was added. After that, the seed culture of Escherichia coli strain SCS110-AF/VTvaf17-SHH was inoculated into a culture flask in the volume of 100 ml. The culture was incubated in an incubator shaker for 16 hours at 30° C. The seed culture was transferred to the Techfors S bioreactor (Infors HT, Switzerland) and grown to a stationary phase. The process was controlled by measuring optical density of the culture at 600 nm. The cells were pelleted for 30 minutes at 5,000-10,000 g. Supernatant was removed, and the cell pellet was re-suspended in 10% (by volume) phosphate buffered saline. The cells were centrifuged again for 30 minutes at 5,000-10,000 g. Supernatant was removed, a solution of 20 mM TrisCl, 1 mM EDTA, 200 g/l sucrose, pH 8.0 was added to the cell pellet in the volume of 1000 ml, and the mixture was stirred thoroughly to a homogenised suspension. Then egg lysozyme solution was added to the final concentration of 100 μg/ml. The mixture was incubated for 20 minutes on ice while stirring gently. Then 2500 ml of 0.2 M NaOH, 10 g/l sodium dodecyl sulphate (SDS) was added, the mixture was incubated for 10 minutes on ice while stirring gently, then 3500 ml of 3 M sodium acetate, 2 M acetic acid, pH 5-5.5 was added, and the mixture was incubated for 10 minutes on ice while stirring gently. The resulting sample was centrifuged for 20-30 minutes at 15,000 g or a greater value. The solution was decanted delicately, and residual precipitate was removed by passing through a coarse filter (filter paper). Then, RNase A (Sigma, USA) was added to the final concentration of 20 μg/ml, and the solution was incubated overnight for 16 hours at room temperature. The solution was then centrifuged for 20-30 minutes at 15,000 g and passed through a 0.45 μm membrane filter (Millipore, USA). Then, ultrafiltration was performed with a 100 kDa membrane (Millipore, USA) and the mixture was diluted to the initial volume with a buffer solution of 25 mM TrisCl, pH 7.0. This manipulation was performed three to four times. The solution was applied to the column with 250 ml of DEAE Sepharose HP (GE, USA), equilibrated with 25 mM TrisCl, pH 7.0. After the application of the sample, the column was washed with three volumes of the same solution and then gene therapy DNA vector VTvaf17-SHH was eluted using a linear gradient of 25 mM TrisCl, pH 7.0, to obtain a solution of 25mM TrisCl, pH 7.0, 1 M NaCl, five times the volume of the column. The elution process was controlled by measuring optical density of the run-off solution at 260 nm. Chromatographic fractions containing gene therapy DNA vector VTvaf17-SHH were joined together and subjected to gel filtration using Superdex 200 (GE, USA). The column was equilibrated with phosphate buffered saline. The elution process was controlled by measuring optical density of the run-off solution at 260 nm, and the fractions were analysed by agarose gel electrophoresis. The fractions containing gene therapy DNA vector VTvaf17-SHH were joined together and stored at −20° C. To assess the process reproducibility, the indicated processing operations were repeated five times. All processing operations for Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A were performed in a similar way.

The process reproducibility and quantitative characteristics of final product yield confirm the producibility and constructability of gene therapy DNA vector VTvaf17-SHH, or VTvaf17-CTNNB1, or VTvaf17-NOG, or VTvaf17-WNT7A on an industrial scale.

Thus, the produced gene therapy DNA vector containing the therapeutic gene can be used to deliver it to the cells of human beings and animals that experience reduced or insufficient expression of protein encoded by this gene, thus ensuring the desired therapeutic effect.

The purpose set in this invention, namely the construction of the gene therapy DNA vectors in order to increase the expression level of SHH, CTNNB1, NOG, and WNT7A genes that combine the following properties:
I) The effectiveness of upregulation of therapeutic genes in eukaryotic cells due to the obtained gene therapy vectors with a minimum length,
II) Possibility of safe use in gene therapy of human beings and animals due to the absence of regulatory elements representing the nucleotide sequences of viral genomes and antibiotic resistance genes in the gene therapy DNA vector,
III) Producibility and constructability in the strains on an industrial scale,
IV) as well as the purpose of the construction of strains carrying these gene therapy

DNA vectors for the production of these gene therapy DNA vectors is achieved, which is supported by the following examples:

### for Item I—Example 1, 2, 3, 4, 5; 6; 7; 8; 9; 10; 11; 12; 13; 14; 15; 16; 17; 18.

### for Item II—Example 1, 2, 3, 4

for Item III and Item IV—Example 19, 20.

### INDUSTRIAL APPLICABILITY

All the examples listed above confirm the industrial applicability of the proposed gene therapy DNA vector based on gene therapy DNA vector VTvaf17 carrying the therapeutic gene selected from the group of SHH, CTNNB1, NOG, and WNT7A genes in order to increase the expression level of these therapeutic genes, method of its production and use, Escherichia coli strain SCS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A carrying gene therapy DNA vector, and method of its production on an industrial scale.

List of Oligonucleotide Sequences:
(1) SHH_F
AGGATCCACCATGCTGCTGCTGGCGAGATGTC
(2) SHH_R
TATAAGCTTTCAGCTGGACTTGACCGCCAT
(3) SHH_SF
TTATCCCCAATGTGGCCGAG
(4) SHH_FR
CTGAGTCATCAGCCTGTCCG
(5) CTNNB_F
ATCGTCGACCACCATGGCTACCCAAGCTGATTTG
(6) CTNNB_R
TTCGGTACCTTACAGGTCAGTATCAAACCAG
(7) CTNNB_SF
ATGACTCGAGCTCAGAGGGT
(8) CTNNB_SR
ATTGCACGTGTGGCAAGTTC
(9) NOG_F
GGATCCACCATGGAGCGCTGCCCCAG
(10) NOG_R
ATAGAATTCTAGCACGAGCACTTGCACT
(11) NOG_SF
GATCTGAACGAGACGCTGCT
(12) NOG_SR
TAGCCCTTTGATCTCGCTCG
(13) WNT_F
ATCGTCGACCACCATGAACCGGAAAGCGCGGCGCT
(14) WNT_R
TTCGGTACCTCACTTGCACGTGTACATCTCCGT
(15) WNT_SF
GCGACAAAGAGAAGCAAGGC
(16) WNT_SR
CTCCTCCAGGATCTTTCGGC

### LIST OF ABBREVIATIONS

VTvaf17—Gene therapy vector devoid of sequences of viral genomes and antibiotic resistance markers (vector therapeutic virus-antibiotic-free)
DNA—Deoxyribonucleic acid
cDNA—Complementary deoxyribonucleic acid
RNA—Ribonucleic acid
mRNA—Messenger ribonucleic acid
bp—base pair
PCR—Polymerase chain reaction
ml—millilitre, μl—microlitre
mm3—cubic millimetre
l—litre
μg—microgram
mg—milligram
g—gram
μM—micromol
mM—millimol
min—minute
s—second
rpm—rotations per minute
nm—nanometre
cm—centimetre
mW—milliwatt
RFU—Relative fluorescence unit
PBS—Phosphate buffered saline

### LIST OF REFERENCES

1. Ahmed, R. P. H., Haider, K. H., Shujia, J., Afzal, M. R., & Ashraf, M. (2010). Sonic Hedgehog Gene Delivery to the Rodent Heart Promotes Angiogenesis via iNOS/Netrin-1/PKC Pathway. PLoS ONE, 5(1), e8576.
2. Benraiss A, Bruel-Jungerman E, Lu G, Economides A N, Davidson B, Goldman S A. Sustained induction of neuronal addition to the adult rat neostriatum by AAV4-delivered NOG and BDNF. Gene Ther. 2012 May;19(5):483-93.
3. Bentzinger C F, von Maltzahn J, Dumont N A, Stark D A, Wang Y X, Nhan K, Frenette J, Cornelison D D, Rudnicki M A. Wnt7a stimulates myogenic stem cell motility and engraftment resulting in improved muscle strength. J Cell Biol. 2014 Apr. 14;205(1):97-111.
4. Botchkarev V A, Botchkareva N V, Nakamura M, Huber O, Funa K, Lauster R, Paus R, Gilchrest B A. NOG is required for induction of the hair follicle growth phase in postnatal skin. FASEB J. 2001 October;15(12):2205-14.
5. Cooper G M, Usas A, Olshanski A, Mooney M P, Losee J E, Huard J. Ex vivo NOG gene therapy inhibits bone formation in a mouse model of postoperative resynostosis. Plast Reconstr Surg. 2009 February;123(2 Suppl):94S-103 S.
6. Cui C Y, Kunisada M, Childress V, Michel M, Schlessinger D. Shh is required for Tabby hair follicle development. Cell Cycle. 2011 Oct. 1;10(19):3379-86.
7. daughter. N Engl J Med 263:839-842.
8. daughter. N Engl J Med 263:839-842.
9. daughter. N Engl J Med 263:839-842.
10. Dong L, Hao H, Liu J, Ti D, Tong C, Hou Q, Li M, Zheng J, Liu G, Fu X, Han W. A Conditioned Medium of Umbilical Cord Mesenchymal Stem Cells Overexpressing Wnt7a Promotes Wound Repair and Regeneration of Hair Follicles in Mice. Stem Cells Int. 2017;2017:3738071.
11. Draft Guideline on the quality, non-clinical and clinical aspects of gene therapy medicinal products, http://www.ema.europa.eu/docs/en_GB/document_library/Scientific_guideline/2015/05/WC500187020.pdf
12. Enshell-Seijffers D, Lindon C, Wu E, Taketo M M, Morgan B A. Beta-catenin activity in the dermal papilla of the hair follicle regulates pigment-type switching. Proc Natl Acad Sci U S A. 2010 Dec. 14;107(50):21564-9.
13. Fiuraskova M, Brychtova S, Kolar Z, Kucerova R, Bienova M. Expression of beta-catenin, p63 and CD34 in hair follicles during the course of androgenetic alopecia. Arch Dermatol Res. 2005 September;297(3):143-6. Epub 2005 Sep. 29.
14. Ghadakzadeh S, Kannu P, Whetstone H, Howard A, Alman B A. β-Catenin modulation in neurofibromatosis type 1 bone repair: therapeutic implications. FASEB J. 2016 September;30(9):3227-37
15. Glaser, Kaplan, Wang, Wilson, Stahl, Shore NOG GENE THERAPY FOR THE PREVENTION OF BMP INDUCED HETEROTOPIC OSSIFICATION IN THE MOUSE: IMPLICATIONS FOR THE TREATMENT OF FIBRODYSPLASIA OSSIFICANS PROGRESSIVA AND OTHER DISORDERS OF ECTOPIC OSSIFICATION IN HUMANS. 48th Annual Meeting of the Orthopaedic Research Society; Poster No: 0493
16. Guideline on the quality, non-clinical and clinical aspects of gene therapy medicinal Products EMA/CAT/80183/2014
17. Hahn J Y, Cho H J, Bae J W, Yuk H S, Kim K I, Park K W, Koo B K, Chae I H, Shin C S, Oh B H, Choi Y S, Park Y B, Kim H S. Beta-catenin overexpression reduces myocardial infarct size through differential effects on cardiomyocytes and cardiac fibroblasts. J Biol Chem. 2006 Oct. 13;281(41):30979-89.
18. Hornstein B D, Roman D, Aŕevalo-Soliz L M, Engevik M A, Zechiedrich L. Effects of Circular DNA Length on Transfection Efficiency by Electroporation into HeLa Cells. Celña V, ed. PLoS ONE. 2016;11(12):e0167537. doi:10.1371/journal.pone.0167537.
19. Ito, Z. Yang, T. Andl et al., “Wnt-dependent de novo hair follicle regeneration in adult mouse skin after wounding,” Nature, vol. 447, no. 7142, pp. 316-320,2007.
20. Kharbanda M, Pilz D T, Tomkins S, Chandler K, Saggar A, Fryer A, McKay V, Louro P, Smith J C, Burn J, Kini U, De Burca A, FitzPatrick D R, Kinning E; DDD Study. Clinical features associated with CTNNB1 de novo loss of function mutations in ten individuals. Eur J Med Genet. 2017 February;60(2):130-135.
21. Lee Y B, Eun Y S, Lee J H, Cheon M S, Park Y G, Cho B K, Park H J. Effects of topical application of growth factors followed by microneedle therapy in women with female pattern hair loss: a pilot study. J Dermatol. 2013 January;40(1):81-3.
22. Li H, Fan L, Zhu S, Shin M K, Lu F, Qu J, Hou L. Epilation induces hair and skin pigmentation through an EDN3/EDNRB-dependent regenerative response of melanocyte stem cells. Sci Rep. 2017 Aug. 4;7(1):7272.
23. Li L, Petrovsky N. Molecular mechanisms for enhanced DNA vaccine immunogenicity. Expert Rev Vaccines. 2016;15(3):313-29
24. Ma H, Marti-Gutierrez N, Park S W, Wu J, Lee Y, Suzuki K, Koski A, Ji D, Hayama T, Ahmed R, Darby H, Van Dyken C, Li Y, Kang E, Park A R, Kim D, Kim S T, Gong J, Gu Y, Xu X, Battaglia D, Krieg S A, Lee D M, Wu D H, Wolf D P, Heitner S B, Belmonte J C I, Amato P, Kim J S, Kaul S, Mitalipov S. Correction of a pathogenic gene mutation in human embryos. Nature. 2017 Aug. 24;548(7668):413-419.
25. Mairhofer J, Grabherr R. Rational vector design for efficient non-viral gene delivery: challenges facing the use of plasmid DNA. Mol Biotechnol. 2008.39(2):97-104
26. McCollum P T, Bush J A, James G, Mason T, O'Kane S, McCollum C, Krievins D, Shiralkar S, Ferguson M W. Randomized phase II clinical trial of avotermin versus placebo for scar improvement. Br J Surg. 2011 July;98(7):925-34.
27. Mullor J L, Sánchez P, Ruiz i Altaba A. Pathways and consequences: Hedgehog signalling in human disease. Trends Cell Biol. 2002 December;12(12):562-9.
28. Paladini R D, Saleh J, Qian C, Xu G X, Rubin L L. Modulation of hair growth with small molecule agonists of the hedgehog signalling pathway. J Invest Dermatol. 2005 October; 125(4): 638-46.
29. Park H J, Lee J, Kim M J, Kang T J, Jeong Y, Um S H, Cho S W. Sonic hedgehog intradermal gene therapy using a biodegradable poly((β-amino esters) nanoparticle to enhance wound healing. Biomaterials. 2012 Dec;33(35):9148-56.
30. Reflection paper on design modifications of gene therapy medicinal products during development/14 Dec. 2011 EMA/CAT/GTWP/44236/2009 Committee for advanced therapies
31. Reis L M, Semina E V. Conserved genetic pathways associated with microphthalmia, anophthalmia, and coloboma. Birth Defects Res C Embryo Today. 2015 June;105(2):96-113.
32. Roncalli J, Renault M-A, Tongers J, et al. Sonic-hedgehog-induced functional recovery after myocardial infarction is enhanced by AMD3100-mediated progenitor-cell mobilization. Journal of the American College of Cardiology. 2011;57(24):2444-2452.
33. Shi F, Hu L, Jacques B E, Mulvaney J F, Dabdoub A, Edge A S. β-Catenin is required for hair-cell differentiation in the cochlea. J Neurosci. 2014 May 7;34(19):6470-9.
34. Shin J A, Lim S M, Jeong S I, Kang J L, Park E M. NOG improves ischemic brain tissue repair and promotes alternative activation of microglia in mice. Brain Behav Immun. 2014 August; 40:143-54.
35. Tosti A, Zaiac M N, Canazza A, Sanchis-Gomar F, Pareja-Galeano H, Alis R, Lucia A, Emanuele E. Topical application of the Wnt/β-catenin activator methyl vanillate increases hair count and hair mass index in women with androgenetic alopecia. J Cosmet Dermatol. 2016 December;15(4):469-474.
36. Tran K A, Zhang X, Predescu D, Huang X, Machado R F, Göthert J R, Malik A B, Valyi-Nagy T, Zhao Y Y. Endothelial β-Catenin Signalling Is Required for Maintaining Adult Blood-Brain Barrier Integrity and Central Nervous System Homeostasis. Circulation. 2016 Jan. 12;133(2):177-86.
37. Tucker A S, Matthews K L, Sharpe P T. Transformation of tooth type induced by inhibition of BMP signalling. Science 1998;282:1136-8.
38. Usami N, Sekido Y, Maeda O, Yamamoto K, Minna J D, Hasegawa Y, Yoshioka H, Imaizumi M, Ueda Y, Takahashi M, Shimokata K. Beta-catenin inhibits cell growth of a malignant mesothelioma cell line, NCI-H28, with a 3p21.3 homozygous deletion. Oncogene. 2003 Sep. 11;22(39):7923-30.
39. Vesell E S. 1960. Symphalangism, strabismus and hearing loss in mother and
40. Vesell E S. 1960. Symphalangism, strabismus and hearing loss in mother and
41. Vesell E S. 1960. Symphalangism, strabismus and hearing loss in mother and
42. von Maltzahn J, Renaud J M, Parise G, Rudnicki M A. Wnt7a treatment ameliorates muscular dystrophy. Proc Natl Acad Sci U S A. 2012 Dec. 11;109(50):20614-9.
43. Wang Z, Havasi A, Gall J M, Mao H, Schwartz J H, Borkan S C. Beta-catenin promotes survival of renal epithelial cells by inhibiting Bax. J Am Soc Nephrol. 2009 September;20(9): 1919-28.
44. Weiss K, Kruszka P, Guillen Sacoto M J, Addissie Y A, Hadley D W, Hadsall C K, Stokes B, Hu P, Roessler E, Solomon B, Wiggs E, Thurm A, Hufnagel R B, Zein W M, Hahn J S, Stashinko E, Levey E, Baldwin D, Clegg N J, Delgado M R, Muenke M. In-depth investigations of adolescents and adults with holoprosencephaly identify unique characteristics. Genet Med. 2018 January;20(1):14-23.
45. Williams J A. Hedgehog and spinal cord injury. Expert Opin Ther Targets. 2005 December;9(6): 1137-45.
46. Xie Z, Wang P, Li Y, Deng W, Zhang X, Su H, Li D, Wu Y, Shen H. Imbalance Between Bone Morphogenetic Protein 2 and NOG Induces Abnormal Osteogenic Differentiation of Mesenchymal Stem Cells in Ankylosing Spondylitis. Arthritis Rheumatol. 2016 February;68(2):430-40.
47. Yeh W H, Chiang H, Rees H A, Edge A S B, Liu D R. In vivo base editing of post-mitotic sensory cells. Nat Commun. 2018 Jun. 5;9(1):2184
48. You J, Nguyen A V, Albers C G, Lin F, Holcombe R F. Wnt pathway-related gene expression in inflammatory bowel disease. Dig Dis Sci. 2008 April;53(4):1013-9.
49. Zhang Y, Wang N, Ma J, Chen X C, Li Z, Zhao W. Expression profile analysis of new candidate genes for the therapy of primary osteoporosis. Eur Rev Med Pharmacol Sci. 2016;20(3):433-40.
50. Application No. US20060105950A1
51. Application No. WO2000031134A1
52. Application No. WO2013040341A2
53. Molecular Biology, 2011, Vol. 45, No. 1, p. 44-55
54. U.S. Pat. No. 9,550,998 B2
55. U.S. Pat. No. 5,843,775A

1. Gene therapy DNA vector based on gene therapy DNA vector VTvaf17 for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia while the gene therapy DNA vector has the coding region of SHH therapeutic gene cloned to gene therapy DNA vector VTvaf17 resulting in gene therapy DNA vector VTvaf17-SHH that has nucleotide sequence SEQ ID No. 1.

2. Gene therapy DNA vector based on gene therapy DNA vector VTvaf17 for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia while the gene therapy DNA vector has the coding region of CTNNB1 therapeutic gene cloned to gene therapy DNA vector VTvaf17 resulting in gene therapy DNA vector VTvaf17-CTNNB1 that has nucleotide sequence SEQ ID No. 2.

3. Gene therapy DNA vector based on gene therapy DNA vector VTvaf17 for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia while the gene therapy DNA vector has the coding region of NOG therapeutic gene cloned to gene therapy DNA vector VTvaf17 resulting in gene therapy DNA vector VTvaf17-NOG that has nucleotide sequence SEQ ID No. 3.

4. Gene therapy DNA vector based on gene therapy DNA vector VTvaf17 for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia while the gene therapy DNA vector has the coding region of WNT7A therapeutic gene cloned to gene therapy DNA vector VTvaf17 resulting in gene therapy DNA vector VTvaf17-WNT7A that has nucleotide sequence SEQ ID No. 4.

5. Gene therapy DNA vector based on gene therapy DNA vector VTvaf17 carrying SHH, CTNNB1, NOG, or WNT7A therapeutic gene as per claim 1, 2, 3, or 4. Said gene therapy DNA vectors are unique due to the fact that each of the constructed gene therapy DNA vectors: VTvaf17-SHH, or VTvaf17-CTNNB1, or VTvaf17-NOG, or VTvaf17-WNT7A as per claim 1, 2, 3, or 4 due to the limited size of VTvaf17 vector part not exceeding 3200 bp has the ability to efficiently penetrate into human and animal cells and express the SHH, or CTNNB1, or NOG, or WNT7A therapeutic gene cloned to it.

6. Gene therapy DNA vector based on gene therapy DNA vector VTvaf17 carrying SHH, CTNNB1, NOG, or WNT7A therapeutic gene as per claim 1, 2, 3, or 4. Said gene therapy DNA vectors are unique due to the fact that each of the constructed gene therapy DNA vectors: VTvaf17-SHH, or VTvaf17-CTNNB1, or VTvaf17-NOG, or VTvaf17-WNT7A as per claim 1, 2, 3, or 4 uses nucleotide sequences that are not antibiotic resistance genes, virus genes, or regulatory elements of viral genomes as structure elements, which ensures its safe use for gene therapy in humans and animals.

7. A method of gene therapy DNA vector production based on gene therapy DNA vector VTvaf17 carrying the SHH, CTNNB1, NOG, and WNT7A therapeutic gene as per claim 1, 2, 3, or 4 that involves obtaining each of gene therapy DNA vectors: VTvaf17-SHH, or VTvaf17-CTNNB1, or VTvaf17-NOG, or VTvaf17-WNT7A as follows: the coding region of the SHH, or CTNNB1, or NOG, or WNT7A therapeutic gene as per claim 1, 2, 3, or 4 is cloned to gene therapy DNA vector VTvaf17, and gene therapy DNA vector VTvaf17-SHH, SEQ ID No. 1, or VTvaf17-CTNNB1, SEQ ID No. 2, or VTvaf17-NOG, SEQ ID No. 3, or VTvaf17-CAT, SEQ ID No. 4, respectively, is obtained, while the coding region of the SHH, or CTNNB1, or NOG, or WNT7A therapeutic gene is obtained by isolating total RNA from the human biological tissue sample followed by the reverse transcription reaction and PCR amplification using the obtained oligonucleotides and cleaving the amplification product by corresponding restriction endonucleases, while cloning to the gene therapy DNA vector VTvaf17 is performed by BamHI and HindIII, or SalI and KpnI, or BamHI and EcoRI restriction sites, while the selection is performed without antibiotics.
at the same time, the following oligonucleotides produced for this purpose are used during gene therapy DNA vector VTvaf17-SHH, SEQ ID No. 1 production for the reverse transcription reaction and PCR amplification:
SHH_F
AGGATCCACCATGCTGCTGCTGGCGAGATGTC,
SHH_R
TATAAGCTTTCAGCTGGACTTGACCGCCAT,
and the cleaving of amplification product and cloning of the coding region of SHH gene to gene therapy DNA vector VTvaf17 is performed by BamHI and HindIII restriction endonucleases,
at the same time, the following oligonucleotides produced for this purpose are used during gene therapy DNA vector VTvaf17-CTNNB1, SEQ ID No. 2 production for the reverse transcription reaction and PCR amplification:
CTNNB1_F
ATCGTCGACCACCATGGCTACCCAAGCTGATTTG,
CTNNB1_R
TTCGGTACCTTACAGGTCAGTATCAAACCAG,
and the cleaving of amplification product and cloning of the coding region of CTNNB 1 gene to gene therapy DNA vector VTvaf17 is performed by SaII and KpnI restriction endonucleases.
at the same time, the following oligonucleotides produced for this purpose are used during gene therapy DNA vector VTvaf17-NOG, SEQ ID No. 3 production for the reverse transcription reaction and PCR amplification:
NOG_F
GGATCCACCATGGAGCGCTGCCCCAG,
NOG_R
ATAGAATTCTAGCACGAGCACTTGCACT,
and the cleaving of amplification product and cloning of the coding region of NOG gene to gene therapy DNA vector VTvaf17 is performed by BamHI and EcoRI restriction endonucleases,
at the same time, the following oligonucleotides produced for this purpose are used during gene therapy DNA vector VTvaf17-WNT7A, SEQ ID No. 4 production for the reverse transcription reaction and PCR amplification:
WNT7A_F
ATCGTCGACCACCATGAACCGGAAAGCGCGGCGCT,
WNT7A_R
TTCGGTACCTCACTTGCACGTGTACATCTCCGT,
and the cleaving of amplification product and cloning of the coding region of WNT7A gene to gene therapy DNA vector VTvaf17 is performed by SalI and KpnI restriction endonucleases.

8. A method of use of the gene therapy DNA vector based on gene therapy DNA vector VTvaf17 carrying SHH, CTNNB1, NOG, and WNT7A therapeutic gene as per claim 1, 2, 3, or 4 for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia that involves transfection of the cells of patient or animal organs and tissues with the selected gene therapy DNA vector carrying the therapeutic gene based on gene therapy DNA vector VTvaf17, or several selected gene therapy DNA vectors carrying therapeutic genes based on gene therapy DNA vector VTvaf17, from the group of constructed gene therapy DNA vectors carrying therapeutic genes based on gene therapy DNA vector VTvaf17 and/or injection of autologous cells of said patient or animal transfected by the selected gene therapy DNA vector carrying therapeutic gene based on gene therapy DNA vector VTvaf17 or several selected gene therapy DNA vectors carrying the therapeutic genes based on gene therapy DNA vector VTvafl 7 from the constructed gene therapy DNA vectors carrying therapeutic genes based on gene therapy DNA vector VTvaf17 into the organs and tissues of the same patient or animal and/or the injection of the selected gene therapy DNA vector carrying therapeutic gene based on gene therapy DNA vector VTvaf17 or several selected gene therapy DNA vectors carrying therapeutic genes based on gene therapy DNA vector VTvaf17 from the group of constructed gene therapy DNA vectors carrying therapeutic genes based on gene therapy DNA vector VTvaf17 into the organs and tissues of the same patient or animal, or the combination of the indicated methods.

9. A method of production of strain for construction of a gene therapy DNA vector as per claim 1, 2, 3, or 4 for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia that involves making electrocompetent cells of Escherichia coli strain SCS110-AF and subjecting these cells to electroporation with gene therapy DNA vector VTvaf17-SHH, or gene therapy DNA vector VTvaf17-CTNNB1, or gene therapy DNA vector VTvaf17-NOG, or gene therapy DNA vector VTvaf17-WNT7A. After that, the cells are poured into agar plates (Petri dishes) with a selective medium containing yeastrel, peptone, 6% sucrose, and 10 μg/m1 of chloramphenicol, and as a result, Escherichia coli strain SCS110-AF/VTvaf17-SHH or Escherichia coli strain SCS110-AF/VTvaf17-CTNNB 1, or Escherichia coli strain SCS110-AF/VTvaf17-NOG, or Escherichia coli strain SCS110-AF/VTvaf17-WNT7A is obtained.

10. Escherichia coli strain SCS110-AF/VTvaf17-SHH obtained as per claim 9 carrying the gene therapy DNA vector VTvaf17-SHH for production thereof allowing for antibiotic-free selection during the production of the gene therapy DNA vector for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia.

11. Escherichia coli strain SCS110-AF/VTvaf17-CTNNB1, obtained as per claim 9 carrying the gene therapy DNA vector VTvaf17-CTNNB1 for production thereof allowing for antibiotic-free selection during the production of the gene therapy DNA vector for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia.

12. Escherichia coli strain SCS110-AF/VTvaf17-NOG, obtained as per claim 9 carrying the gene therapy DNA vector VTvaf17-NOG for production thereof allowing for antibiotic-free selection during the production of the gene therapy DNA vector for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia.

13. Escherichia coli strain SCS110-AF/VTvaf17-WNT7A, obtained as per claim 9 carrying the gene therapy DNA vector VTvaf17-WNT7A for production thereof allowing for antibiotic-free selection during the production of the gene therapy DNA vector for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia.

14. A method of production on an industrial scale of gene therapy DNA vector based on gene therapy DNA vector VTvaf17 carrying the SHH, or CTNNB1, or NOG, or WNT7A therapeutic gene as per claim 1, 2, 3, or 4 for treatment of diseases associated with disorders of tissue regeneration, wound healing, hair growth, pigmentation, and colouring, formation and maturation of follicles, and cells differentiation and growth process leading to reduced activity of follicles, including in case of alopecia, autoimmune diseases, hereditary and acquired pathological conditions, and for accelerated wound healing, hair cover restoration, and prevention and inhibition of alopecia that involves production of gene therapy DNA vector VTvaf17-SHH, or gene therapy DNA vector VTvaf17-CTNNB1, or gene therapy DNA vector VTvaf17-NOG, or gene therapy DNA vector VTvaf17-WNT7A by inoculating a culture flask containing the prepared medium with seed culture selected from Escherichia coli strain CS110-AF/VTvaf17-SHH, or Escherichia coli strain SCS110-AF/VTvaf17-CTNNB1, or Escherichia coli strain SCS110-AF/VTvaf17-NOG, or Escherichia coli strain SCS110-AF/VTvaf17-WNT7A then the cell culture is incubated in an incubator shaker and transferred to an industrial fermenter, then grown to a stationary phase, then the fraction containing the target DNA product is extracted, multi-stage filtered, and purified by chromatographic methods.
